(19) United States
(12) Patent Application Publication
(10) Pub. No.: US 2025/0249045 A1
(43) Pub. Date: Aug. 7, 2025

(54) COMPOSITIONS AND METHODS RELATED TO EXTRACELLULAR VESICLE THERAPEUTIC DELIVERY PLATFORM

(71) Applicant: North Carolina State University, Raleigh, NC (US)

(72) Inventors: Ke Cheng, Raleigh, NC (US); Kristen Popowski, Raleigh, NC (US); Mengrui Liu, Raleigh, NC (US); Zhenzhen Wang, Raleigh, NC (US)

(21) Appl. No.: 18/855,146

(22) PCT Filed: Apr. 6, 2023
(86) PCT No.: PCT/US2023/017756

§ 371 Date: Oct. 8, 2024

Related U.S. Application Data
(60) Provisional application No. 63/328,844, filed on Apr. 8, 2022.

(51) Int. Cl. A61K 35/42; A61K 9/00; A61K 9/127; A61K 9/50; A61K 38/00; A61K 39/00; A61P 31/12; A61P 35/00; C07K 14/005; C07K 14/54; C12N 5/071; C12N 15/88

(52) U.S. Cl. CPC A61K 35/42; A61K 9/0075; A61K 9/0078; A61K 9/127; A61K 9/5068; A61P 31/12; A61P 35/00; C07K 14/005; C07K 14/5434; C12N 5/0688; C12N 15/88; A61K 38/00; A61K 2039/544; A61K 2039/55555; C12N 2770/20034

(57) ABSTRACT

The present disclosure provides compositions and methods related to engineered extracellular vesicles (EVs). In particular, the present disclosure provides a novel delivery platform for administering therapeutic agents to a subject using engineered EVs. As provided herein, EVs can be used to deliver biologically active cargo (e.g., mRNA, tumor antigens, small molecule drugs) to a subject to treat and/or prevent disease (e.g., viral infection, cancer, etc.).

### CROSS REFERENCE TO RELATED APPLICATIONS

This application claims priority to and the benefit of U.S. Provisional Patent Application No. 63/328,844 filed Apr. 8, 2022, which is incorporated herein by reference in its entirety for all purposes.

### FIELD

The present disclosure provides compositions and methods related to engineered extracellular vesicles (EVs). In particular, the present disclosure provides a novel delivery platform for administering therapeutic agents to a subject using engineered EVs. As provided herein, EVs can be used to deliver biologically active cargo (e.g., mRNA, tumor antigens, small molecule drugs) to a subject to treat and/or prevent disease (e.g., viral infection, cancer, etc.).

### BACKGROUND

Formulating drugs for inhaled delivery may improve their efficacy and bioavailability in the lung, but their biodistribution after inhalation is yet to be elucidated. Although inhalation delivery is attractive due to its local, noninvasive, and highly absorptive properties, drug formulation with optimized physiochemical parameters remains the key obstacle. A possible solution is colloidal drug delivery systems, which help overcome poor drug solubility and hydrophobicity by providing protection through nanoparticle encapsulation. Plus, nanoparticles retained in the lung prolong drug release in the highly vascularized pulmonary and bronchial circulations. Recently, lipid nanoparticles have been successful as drug delivery vesicles for vaccines most notably in the liposome-encapsulated COVID-19 mRNA vaccines, which are delivered intramuscularly. However, the lung has sophisticated pulmonary defense mechanisms and surfactants that protect it against inhaled particulates and microbes. Therefore, nanoparticle drug delivery systems must be optimized to overcome these inherent obstacles to be administered via inhalation.

Exosomes have emerged with promising applications in nanotechnology and nanomedicine. They're nanosized extracellular vesicles secreted by numerous cell types and found in almost all biological fluids. Initially regarded as cellular debris, exosomes are now understood to have potent roles in autocrine and paracrine signaling. Originating from the endosomal system and shedding from the plasma membrane, exosomes contain unique cocktails of RNA, protein, and lipid cargo with unique parent-cell signatures. Lung-derived exosomes can be utilized as sophisticated drug delivery systems that offer cargo components and membrane features tailored to the lung microenvironment. Additionally, exosomes can be synthetically supplemented to enhance cellular targeting and therapeutic efficacy. The combination of vesicle derivation and supplementation allows for a customizable nanoparticle delivery platform that can be utilized across many major lung diseases.

### SUMMARY

Embodiments of the present disclosure include a composition comprising a plurality of engineered extracellular vesicles (EVs), wherein the plurality of EVs comprise: (i) at least one membrane-associated protein on the surface of the plurality of EVs; and/or (ii) at least one therapeutic agent loaded into the plurality of EVs.

In some embodiments, the plurality of EVs are derived from a cell. In some embodiments, the plurality of EVs are derived from a cell selected from the group consisting of: HeLa cells, HEK293 cells, HEK293 derived cells, Vero cells, CHO cells, CHO-K1 cells, CHO-derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, and RK cells.

In some embodiments, the plurality of EVs are derived from a lung spheroid cell (LSC).

In some embodiments, the plurality of EVs comprise liposomes. In some embodiments, the plurality of EVs comprise exosomes.

In some embodiments, the plurality of EVs are from about 30 nm to about 1000 nm in diameter. In some embodiments, the plurality of EVs comprise an average size from about 100 nm to about 200 nm in diameter.

In some embodiments, the at least one membrane-associated protein on the surface of the plurality of EVs comprises a viral-specific protein, or a derivative or fragment thereof. In some embodiments, the viral-specific protein comprises an antigenic epitope or derivative or fragment thereof capable of stimulating an immune response in a subject. In some embodiments, the viral-specific protein comprises a coronavirus Spike protein (S protein), or a derivative or fragment thereof. In some embodiments, the viral-specific protein comprises a receptor binding domain (RBD) of a coronavirus Spike protein (S protein), or a derivative or fragment thereof, capable of binding Angiotensin-converting enzyme 2 (ACE2).

In some embodiments, the at least one therapeutic agent loaded into the plurality of EVs comprises mRNA encoding an antigenic epitope or derivative or fragment thereof capable of stimulating an immune response in a subject. In some embodiments, the mRNA encodes an antigenic epitope or a derivative or fragment thereof capable of stimulating an immune response in a subject from a coronavirus Spike protein (S protein), or a derivative or fragment thereof. In some embodiments, the mRNA encodes an antigenic epitope or a derivative or fragment thereof capable of stimulating an immune response in a subject from a receptor binding domain (RBD) of a coronavirus Spike protein (S protein), or a derivative or fragment thereof, capable of binding Angiotensin-converting enzyme 2 (ACE2).

In some embodiments, the at least one therapeutic agent loaded into the plurality of EVs comprises at least one mRNA encoding a tumor-associated antigen (TAA). In some embodiments, the TAA is selected from the group consisting of: MAGE-C1, MAGE-C2, MAGE-C3, MAGE-A3, NY-SEO-1, survivin, 5 T4, MUC1, PSA, PSCA, PSMA, STEAP1, PAP, tyrosinase, GP100, and CT7.

In some embodiments, the at least one therapeutic agent loaded into the plurality of EVs comprises at least one mRNA encoding an immunostimulant. In some embodiments, the immunostimulant is selected from the group consisting of: IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, INF-gamma, GM CSF, G-CSF, M-CSF, LT-βor TNF-α, OX40L, CD40L, and CD7.

In some embodiments, the immunostimulant is IL-12.

In some embodiments, the at least one therapeutic agent loaded into the plurality of EVs comprises at least one small molecule. In some embodiments, the at least one small molecule is an anti-cancer drug. In some embodiments, the anti-cancer drug is selected from the group consisting of: a kinase inhibitor, an ALK inhibitor, a c-Met inhibitor, an EGFR inhibitor, an FLT3 inhibitor, a VEGFR/FGFR/PDGFR inhibitor, a TRK inhibitor, Bcr-Abll inhibitor, a BTK inhibitor, a JAK inhibitor, a BRAF/MEK/ERK inhibitor, a CDK inhibitor, a PI3K/AKT/mTOR inhibitor, an EZH2 inhibitor, an HDAC inhibitor, an IDH1/2 inhibitor, and a BCL-2 inhibitor. In some embodiments, the anti-cancer drug is a VEGFR/FGFR/PDGFR inhibitor selected from the group consisting of: nintedanib, sorafenib, sunitinib, lenvatinib, pazopanib, axitinib, cabozantinib, tivozanib, apatinib, anlotinib, fruquintinib, erdafitinib, pemigatinib, avapritinib, imatinib, regorafenib, ripretinib, cediranib, dovitinib, motesanib, crenolanib, lucitanib, vactosertib, vandetanib, selpercatinib, pralsetinib, sulfatinib, and brivanib.

In some embodiments, loading of the at least one therapeutic agent comprises encapsulating the at least one therapeutic agent in the EV membrane and/or encapsulating the at least one therapeutic agent within the lumen of the EV.

In some embodiments, the composition further comprises at least one pharmaceutically-acceptable excipient or carrier.

Embodiments of the present disclosure also include a method of preventing and/or treating a viral infection comprising administering any of the compositions described herein to a subject. In some embodiments of the method, the virus is a coronavirus. In some embodiments, the coronavirus is selected from the group consisting of 229E, NL63, OC43, HKU1, MERS-CoV, SARS-CoV, and SARS-CoV-2, or any variants thereof.

Embodiments of the present disclosure also include a method of treating cancer comprising administering any of the compositions described herein to a subject. In some embodiments of the method, the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration.

In some embodiments of the method, the composition is administered via nebulization to lung tissue.

### BRIEF DESCRIPTION OF THE DRAWINGS

FIGS 1A-1H: Lung-derived exosomes have superior distribution in the bronchioles and parenchyma. (a) Schematic showing protein loading into lung-derived exosomes (RFP-Exo) and liposomes (RFP-Lipo), nebulization administration, ex vivo lung tissue clearing, and 3D imaging by LSFM. Created with BioRender.com. (b) TEM images of RFP-Exo and RFP-Lipo; scale bar=50 nm. (c) Immunoblot of RFP in exosome and liposome lysate. (d) Representative immunostaining images of lung parenchymal cells for RFP (red) and DAPI (bluc); scale bar=50 μm. (e) Quantification of RFP-Exo and RFP-Lipo pixel intensity normalized to nuclei in lung parenchymal cell images; n=6 per group. (f) LSFM images of cleared mouse lungs after RFP-Exo and RFP-Lipo nebulization; scale bar=1000 μm. (g) Quantification of the integrated density of RFP normalized to the whole lung area; n=74 per group. (h) Quantification of the integrated density of RFP normalized to segmented bronchiole and parenchymal regions from whole lung images; n=74 per group.

FIGS. 2A-2F: Nebulized lung-derived exosomes have superior distribution and retention in the murine lung. (a) Schematic of mRNA and protein loading, nebulization administration, and ex vivo histology. Created with BioRender.com. (b) Representative ex vivo images of mouse lungs after mRNA and protein loaded Lung-Exo, HEK-Exo, and Lipo nebulization. (c) Quantification of the integrated density of GFP and RFP fluorescence in ex vivo mouse lungs; n=1 per group. (d) Quantification of the integrated density of GFP and RFP fluorescence in ex vivo mouse lungs 24 hours after nebulization; n=3 per group. (e) Immunoblots of GFP and RFP in mouse lung lysate. (f) Quantification of immunoblots normalized to β-actin; n=3 per group.

FIGS. 3A-3D: Lung-derived exosomes have superior delivery of mRNA and protein to the bronchioles and parenchyma. (a) Representative immunostaining images of whole lung, tracheal, bronchiole, and parenchymal sections for GFP (green), RFP (red) and DAPI (blue); scale bar=1000 μm in whole lung images; scale bar=100 μm in trachea and bronchioles sections; scale bar=1 μm in parenchyma sections. (b) Quantification of Lung-Exo, HEK-Exo, and Lipo pixel intensity normalized to nuclei in tracheal sections; n=9 per group. (c) Quantification of Lung-Exo, HEK-Exo, and Lipo pixel intensity normalized to nuclei in bronchiole sections; n=9 per group. (d) Quantification of Lung-Exo, HEK-Exo, and Lipo pixel intensity normalized to nuclei in parenchymal sections; n=9 per group.

FIGS. 4A-4G: Distribution of lung-derived exosomes via dry powder inhalation in African green monkeys. (a) Schematic of mRNA and protein loaded lung-derived exosome lyophilized, encapsulation, non-human primate DPI administration, and ex vivo histology. Created with BioRender.com. (b) Quantification of the integrated density of GFP and RFP fluorescence in ex vivo primate lungs 24 hours and 1 week after dry-powder inhalation; n=1 per group. (c) Schematic showing upper respiratory tissue sectioning for nasal (n), sinus(s), tongue (t), and throat (th) sections and lower respiratory tissue sectioning for tracheal (tr), bronchial (b), and parenchymal (p) sections. Created with BioRender.com. (d) Ex vivo images of primate head cross-sections and lungs 24 hours and 1 week after lyophilized Lung-Exo via dry-powder inhalation. (e) Representative immunostaining images of nasal cavity, sinus, tongue, throat, trachea, bronchioles, and parenchyma sections for GFP (green), RFP (red) and DAPI (blue); scale bar=100 μm in representative images; scale bar=1 μm in parenchyma sections. (f) Quantification of Lung-Exo GFP pixel intensity normalized to nuclei in nasal cavity, sinus, tongue, throat, trachea, bronchioles, and parenchyma sections; n=5 per group. (g) Quantification of Lung-Exo RFP pixel intensity normalized to nuclei in nasal cavity, sinus, tongue, throat, trachea, bronchioles, and parenchyma sections; n=5 per group.

FIGS. 5A-5I: Lung-derived exosomes are room-temperature stable and distributable in dry powder formulation in the murine lung. (a) Schematic of mRNA and protein loaded lung-derived exosome lyophilization, encapsulation, rodent DPI administration, and ex vivo histology. Created with BioRender.com. (b) Heat maps of RFP leakage from Lung-Exo, HEK-Exo, and Lipo detected by ELISA; n=2 per group. (c) Representative AFM height (i), amplitude (ii) and phase (iii) images of Lung-Exo; scale=50 nm. (d) Quantification of the height and diameter of Lung-Exo, HEK-Exo, and Lipo from AFM images; n=9 per group. (e) TEM images of Lung-Exo at frozen (Frozen) or room (Lyophilized) temperatures; scale bar=50 nm. (f) Ex vivo images of mouse lungs that received fresh lyophilized (0 days) and 28-day-old lyophilized Lung-Exo via dry-powder inhalation after 24 hours. (g) Quantification of the integrated density of GFP and RFP fluorescence in ex vivo mouse lungs 24 hours after fresh (Fresh-Lyo) and 28-day-old (28-Day Lyo) dry-powder inhalation; n=3 per group. (h) Quantification of the integrated density of GFP and RFP fluorescence in ex vivo mouse lungs 24 hours after nebulization and fresh (Fresh-Lyo) dry-powder inhalation; n=3 per group. (i) Quantification of the integrated density of GFP and RFP fluorescence in ex vivo mouse lungs 24 hours after nebulization and 28-day-old (28-Day Lyo) dry-powder inhalation; n=3 per group.

FIGS. 6A-6H: Dry powder inhalation of S protein-loaded lung-derived exosomes bas greater therapeutic efficacy than its synthetic counterpart. (a) Schematic of S protein mRNA loading into lung-derived exosomes, dry powder formulation, inhaled vaccine delivery doses, and antibody production against SARS-CoV-2 spike protein. Created with BioRender.com. (b) TEM images of S-Exo and S-Lipo at room temperature; scale bar=50 nm. (c) NTA size distribution analysis. (d) Quantification of NTA size distribution analysis of the average mean±standard error of five replicates; n=1 per group. (e) Immunoblots of S protein in mouse lung lysate. (f) Quantification of immunoblots normalized to β-actin; n=3 per group. (g) Anti-spike IgG antibody titer from murine BALF detected by ELISA; n=6 per group. (h) Anti-spike SIgA antibody titer from murine NPLF detected by ELISA; n=6 per group.

FIGS. 7A-7H: Nebulized RBD-Exo triggered superior immune responses over RBD-Lipo in mice. (a) Schematic illustrating the modification of LSC-Exo with RBD to generate RBD-Exo. (b) SDS-PAGE gel of RBD and RBD-PEG-DSPE. Transmission electron microscopy (TEM) images of RBD-Exo (c) and RBD-Lipo (d). RBD was detected using gold nanoparticle-labeled secondary antibodies with diameters of 10 nm. (e). Schematic showing animal study design. (f) RBD-specific IgG antibody titer from murine serum detected by ELISA after two doses of RBD-Exo or RBD-Lipo immunizations. RBD-specific IgA antibody titer from BALF (g) and NPLF (h) detected by ELISA after two doses of RBD-Exo or RBD-Lipo immunizations. Data are mean±s.d. n=3. Statistical analysis was performed by one-way ANOVA test with Bonferroni correction. ns indicates no significance.

FIGS. 8A-8H: Nebulized IL12-Exo induced superior tumor accumulation and immunotherapy over IL12-Lipo in mice. (a) Schematic illustrating the construction of IL 12-Exo, and lung delivery of nebulized IL12-Exo followed with local IL12 protein expression in mice. (b) NTA size distribution analysis with TEM images of IL 12-Exo and IL 12-Lipo at room temperature, scale bar=100 nm. (c) Quantified distribution analysis of IL12-Exo and IL12-Lipo in ex vivo mouse organs 24 hours after inhalation of nebulized DiD labeled IL 12-Exo and IL12-Lipo, n=3-4 biological independent mice. (d) Representative immunostaining images of IL 12-Exo and IL 12-Lipo (grey), Macrophages (red), LL/2 tumor cells (Green) and DAPI (blue) in lungs 24 h after inhalation of nebulized IL12-Exo and IL 12-Lipo; scale bar=100 μm. The white arrows indicated the uptake of IL 12-Exo and IL 12-Lipo by LL/2, while the yellow arrows indicated the uptake of IL 12-Exo and IL12-Lipo by Macrophages. (e) Quantification analysis of IL 12-Exo and IL 12-Lipo uptake efficacy in total cells, tumor cells, macrophages, epithelial cells of lungs by flow cytometry; n=3-6 biological independent mice. (f) Uptake percentage of IL12-Exo and IL12-Lipo in different lung cells from FIG. 12E. (g) Tumor growth curves of LL/2 lung tumor in each treatment group (n=6 biological independent mice). (h) Body weight of mice in each treatment group (n=6 biological independent mice). Significant differences were assessed in (c) and (e) using t test, in (g) and (h) using one-way or two-way ANOVA with Bonferroni's multiple comparisons. Results are presented as mean (SD). *p<0.05, **p<0.01, ***p<0.001, ****p<0.0001, ns, not significant.

FIGS. 9A-9H: Nintedanib-loaded exosomes can function as drug delivery carriers to inhibit human idiopathic pulmonary fibrosis lung fibroblasts. (a) Schematic depicting Nintedanib loading in exosomes. (b) Entrapment efficiency of Nin-loaded LSC Exo with increasing exosome ratio to drug, n=5. (c) Entrapment efficiency of Nin-loaded LSC and HEK Exo, n=3. (d) TEM images of Unloaded and Nin-loaded LSC and HEK Exo, scale bar=0.05 μm. (e) NTA size distribution analysis before and after loading Nintedanib into LSC and HEK Exo, n=3. (f) Representative western blot images and relative densitometric bar graphs of phosphorylated PDGFR to total receptor expression in IPF lung fibroblasts following treatment with Nin or Nin-loaded LSC or HEK Exo for 30 min and further stimulation with PDGF for 20 min in the respective wells (values normalized to β-actin), n=3. (g) Representative western blot images of phosphorylated Akt to total Akt expression in IPF lung fibroblasts following treatment with Nin or Nin-loaded LSC or HEK Exo for 30 min and further stimulation with PDGF for 20 min in the respective wells. (h) IPF lung fibroblast proliferation determined by BrdU incorporation. Absorbance was measured 72 h after treatment with Nin or Nin-loaded LSC or HEK Exo for 30 min and further stimulation with PDGF at 450 nm, n=4. Significant differences were assessed in (b) using t test and in (f) and (h) using one-way ANOVA with Bonferroni's multiple comparisons. Results are presented as mean (SD). *p<0.05, **p<0.01, ***p<0.001.

FIGS. 10A-10D: Characterization of native exosomes and liposomes. (a) TEM images of native Lung-Exo, HEK-Exo, and Lipo. (b) SDS-PAGE and immunoblot of CD63 in exosome and liposome lysate. (c) NTA size distribution analysis and mode nanoparticle diameters. (d) Quantification of NTA size distribution analysis.

FIGS. 11A-11G: Exosomes maintain higher mRNA translation and protein expression than liposomes in vitro. (a) Schematic of mRNA and protein loading into exosomes and liposomes. Created with BioRender.com. (b) TEM images of GFP-encoding mRNA and RFP protein loaded Lung-Exo, HEK-Exo, and Lipo; scale bar=100 nm. (c) Immunoblot of RFP in exosome and liposome lysate. (d) NTA size distribution analysis. (e) Quantification of NTA size distribution analysis of the average mean±standard error of five replicates; n=1 per group. (f) Representative immunostaining images of lung parenchymal cells for GFP (green), RFP (red), and DAPI (blue); scale bar=100 μm. (g) Quantification of Lung-Exo, HEK-Exo, and Lipo pixel intensity normalized to nuclei in lung parenchymal cells; n=3 per group.

FIG. 12: Autofluorescence of murine control organs. Ex vivo images of mouse lungs after native Lang-Exo, HEK-Exo, and Lipo nebulization. Integrated densities of GFP and RFP were used to normalize integrated density values of loaded Lung-Exo, HEK-Exo, and Lipo groups (see FIG. 13).

FIGS. 13A-13B: Biodistribution of mRNA and protein from nanoparticles in murine major organs. (a) Ex vivo images of mouse heart, liver, kidney, spleen, GI, and brain 24 hours after loaded Lung-Exo, HEK-Exo, and Lipo nebulization. (b) Quantification of the integrated density of GFP and RFP fluorescence in ex vivo mouse heart, liver, kidney, spleen, GI, and brain normalized to native nanoparticle controls (see FIG. 12).

FIGS. 14A-14C: Particle size distribution produced by DPI. Total cumulative and differential particle counts of Lung-Exo (a), HEK-Exo (b), and Lipo (c) distributed by DPI.

FIGS. 15A-15C: Evaluation of particle size distribution produced by DPI. Cumulative and differential particle count evaluation of LSC-Exo (a), HEK-Exo (b) and Lipo (c) distributed by DPI.

FIG. 16: mRNA and protein fluorescence in simian immunostained images. Representative split-channel immunostaining images of nasal cavity, sinus, tongue, throat, trachea, bronchioles, and parenchyma sections for GFP (green), RFP (red) and DAPI (blue); scale bar=100 μm.

FIG. 17: mRNA and protein fluorescence in simian upper and lower respiratory tissues. Quantification of mRNA and protein fluorescence in simian upper and lower respiratory tissues 24 hours and 1 week after lyophilized Lung-Exo via dry-powder inhalation.

FIG. 18: Autofluorescence of simian upper and lower respiratory tissues. Representative immunostaining images of nasal cavity, sinus, tongue, throat, trachea, bronchioles, and parenchyma sections for GFP (green), RFP (red) and DAPI (blue); scale bar=100 μm.

FIG. 19: RFP ELISA Standard Curve. Standard curve of RFP concentrations in duplicates. Interpolation of the standard curve is represented by the solid line. The 95% confidence interval is represented by the dashed line.

FIGS. 20A-20B: Morphology of HEK-Exo and Lipo at frozen and room temperatures. TEM images of HEK-Exo (a) and Lipo (b) at frozen (Frozen) or room (Lyophilized) temperatures; scale bar=50 nm.

FIGS. 21A-21B: Membrane integrity of HEK-Exo and Lipo. Representative AFM height (i), amplitude (ii), and phase (iii) images of HEK-Exo (a) and Lipo (b) across fresh, lyophilized, and reconstituted formulations.

FIGS. 22A-22C: Distribution of Lung-Exo. AFM images of fresh (a), lyophilized (b) and reconstituted (c) Lung-Exo across fresh, lyophilized, and reconstituted formulations.

FIGS. 23A-23C: Distribution of HEK-Exo. AFM images of fresh (a), lyophilized (b) and reconstituted (c) HEK-Exo across fresh, lyophilized, and reconstituted formulations.

FIGS. 24A-24C: Distribution of Lipo. AFM images of fresh (a), lyophilized (b) and reconstituted (c) Lipo across fresh, lyophilized, and reconstituted formulations.

FIGS. 25A-25C: Cross-section measurements of Lung-Exo, HEK-Exo, and Lipo. Representative AFM images of Lung-Exo (a), HEK-Exo (b), and Lipo (c) across fresh, lyophilized, and reconstituted formulations. Cross-section measurements were repeated on nine singular exosomes or liposomes to obtain height and diameter measurements.

FIGS. 26A-26G: Lung-derived exosomes efficiently penetrate mucus. (a) Schematic of DiD labeling and administration to an air-liquid interface transwell system. Created with BioRender.com. (b) Brightfield images of human bronchial epithelial cells on the transwell membrane and lung parenchymal cells on the transwell well; scale bar=10 μm. (c) Representative immunostaining images of transwell membranes and transwell wells for MUC5b (green), DiD (red), and DAPI (blue); scale bar=10 μm. (d) Quantification of Lung-Exo, HEK-Exo, and Lipo pixel intensity normalized to nuclei in lung parenchymal cells; n=3 per group. (c) Percentage of DiD⁺ lung parenchymal cells; n=3 per group. (f) Quantification of Lung-Exo, HEK-Exo, and Lipo pixel intensity normalized to nuclei in human bronchial epithelial cells; n=3 per group. (g) Percentage of DiD⁺ human bronchial epithelial cells; n=3 per group.

FIGS. 27A-27D: Characterization of extrafacial expression of ACE2 on LSC-Exo. (a) TEM images of LSC-Exo and HEK-Exo. Scale bar: 200 μm. (b) Measurements of size distribution of LSC-Exo and HEK-Exo via NTA. (c) Quantification of ACE2 expression on LSC-Exo and HEK-Exo by flow cytometry. n=3. (d) Western blot and quantification analysis of ACE2 levels on LSC-Exo and HEK-Exo. n=3. Data are mean±s.d. A two-tailed, unpaired Student's t-test was performed for statistical analysis.

FIGS. 28A-28G: LSC-Exo prevents the entry of SARS-CoV-2 pseudovirus. (a) ELISA analysis of the binding affinity between rhACE2 with RBD in the presence of LSC-Exo or HEK-Exo. n=3. (b) Schematic depiction of cell-based neutralization assay. (c) SARS-CoV-2 pseudovirus neutralization analysis of LSC-Exo, HEK-Exo, or rhACE2 in A549 cells expressing ACE2, determined by GFP fluorescence intensity. n=3. (d) Flow plots of SARS-CoV-2 pseudovirus-infected A549 cells that inhibited by LSC-Exo, HEK-Exo, or rhACE2 and its corresponding quantification analysis. n=3. Ex-vivo imaging (e) and quantification analysis (f) of lung from mice inoculated with SARS-CoV-2 pseudovirus. n=3. (g) Quantification analysis of GFP reporter signals in trachea/bronchioles and parenchyma. n=14-20. Data are mean±s.d. Statistical analysis was performed by one-way ANOVA with Bonferroni correction.

FIGS. 29A-29L: Protective effect of LSC-Exo against authentic SARS-CoV-2 infection in Syrian hamsters. (a) Time courses of LSC-Exo inhalation, viral challenge, and measurements. (b) Changes in body weight of hamsters over 1-week post-challenge. n=5. (c) Viral RNA in oral swabs (OS) from hamsters treated with LSC-Exo or PBS. n=5. (d) Viral RNA in bronchoalveolar lavage (BAL) fluid from hamsters treated with LSC-Exo or PBS at 7 days post-challenge. n=5. (e) RNAscope images revealing regional distribution and viral RNA levels in hamster lungs. Immunohistochemistry analysis of SARS-N protein in lung tissues of hamsters. Scale bar, 50 μm. (f) H&E images of representative lung sections of hamsters. Scale bar, 500 μm. (g) Spider web plot displaying histopathological scoring of lung damage, normalized to sham control (green). (h) Masson's trichrome staining of lung sections of hamsters. Scale bar, 500 μm. (i) Quantification analysis of positive SARS-N cell percentages in lungs of hamster. n=15. (j) Ashcroft scoring analysis of lung fibrosis from challenged hamsters that performed blindly. n=5. Viral genomic RNA levels (k) and sgRNA levels (1) in tissues of hamsters with PBS or LSC-Exo treatment. n=5. Data are mean±s.d. Statistical analysis was performed by two-way ANOVA with Tukey's multiple comparisons (b, c, k and l) or two-tailed, unpaired Student's t-test (d, i and j).

### DETAILED DESCRIPTION

Therapeutics have been developed to combat diseases in the lung, but are limited by low pulmonary bioavailability, a failure to fully restore lung function, freezing storage temperatures, and healthcare professional administration. To circumvent such limitations, experiments were conducted to developed room-temperature-stable inhalable extracellular vesicles (EV) or exosomes (Exo) as drug carriers to the lung. Those EVs are deliverable to the lungs of rodents and nonhuman primates (NHP) by jet nebulization and dry powder inhalation through clinically-available devices. EVs from lung cells or generic cell lines such as HEK cells outperform liposome (LNP) counterparts in distribution, retention, and cargo delivery in the lung. Two examples provided herein include the use of inhalable EV to deliver vaccines for SARS-CoV-2 and cancer.

Coronavirus 2 (SARS-CoV-2) spike(S) protein encoding mRNA loaded Lung-Exo (S-Exo) elicited greater immunoglobulin G (IgG) and secretory immunoglobulin A (SIgA) responses than its spike protein loaded liposome (S-Lipo) counterpart. In lung carcinoma mouse models, Exo isolated from HEK cells (HEK-Exo) showed increased lung accumulation with higher tumor cell selectivity compared to that of liposome. Further, IL12 mRNA loaded HEK-Exo (IL12-Exo) induced overwhelming tumor growth inhibition than IL12 mRNA loaded liposomes (IL12-Lipo). A small molecule drug, Nintedanib (an anti-fibrosis drug), was loaded into the exosome for drug delivery. Therefore, embodiments of the present disclosure demonstrate that exosomes are excellent carriers for therapeutic agents, including but not limited to, mRNA, proteins, and small molecules.

As described further herein, embodiments of the present disclosure demonstrate the biodistribution of inhaled exosomes and liposomes in mouse and nonhuman primate (NHP) lungs. Further, exosomal and liposomal mRNA and protein cargo are stable and maintain biological function upon jet nebulization, dry powder formulation, and dry powder inhalation. Experimental data demonstrated that nebulized lung-derived exosomes most efficiently evade mucoadhesion, while maintaining higher mRNA translation, protein expression, and overall cargo retention and distribution in the lung than HEK-exosome and liposome counterparts. Lung-derived exosomes had the highest nanoparticle delivery to the bronchioles and parenchyma, suggesting that nanoparticle phenotypes that are native to the lung microenvironment have enhanced cellular targeting and bioavailability within the lung.

For the first time, embodiments of the present disclosure demonstrated that dry powder formulation of lung-derived exosomes is room-temperature stable and is compatible with clinically-used DPI devices for at-home administration. The Chlorocebus sabaeus NHP model most closely replicates the human airway and respiratory physiology, and exosome delivery was verified in the upper and lower respiratory tracts through DPI administration. Lung-derived exosomes were retained in the primate lung 1-week after a single dry powder inhalation, delivering functional mRNA and protein exosomal cargo from the nose to the deep lung. Lung-derived exosomes are functional an inhaled therapeutic for both upper and lower respiratory diseases.

Lung-derived exosomes offer a unique nanoparticle drug delivery system, with enhanced bioavailability. As described further herein, lung-derived exosomes outperform their HEK-exosome and liposome counterparts in distribution, retention, and cargo delivery in the lung. Additionally, lung-derived exosomes have enhanced therapeutic efficacy for pulmonary disease applications. S protein encoding mRNA remains effective after one month of room temperature storage when lyophilized. As an inhaled vaccine, S protein loaded lung-derived exosomes elicited stronger immune responses than its synthetic counterpart, emphasizing the therapeutic advantages of biological nanoparticles for inhaled vaccines. Furthermore, RBD conjugated lung-derived exosomes induced a superior immunogenicity as the nebulization vaccine over RBD conjugated liposome. In exemplary embodiments, lung-derived exosomes can serve as an mRNA and protein drug delivery vesicle tailored for lung diseases.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” a composition as used herein refers to providing a composition of the present disclosure to a subject in need of treatment (e.g., antiviral treatment). The compositions of the present disclosure may be administered by oral, parenteral (e.g., intramuscular, intraperitoneal, intravenous, ICV, intracisternal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition in the patient (e.g., viral infection). The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

Formulations suitable for nebulizing administration include, for example, aqueous sterile injection solutions, which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The term “culturing” as used herein refers to growing cells or tissue under controlled conditions suitable for survival, generally outside the body (e.g., ex vivo or in vitro). The term includes “expanding,” “passaging,” “maintaining,” etc. when referring to cell culture of the process of culturing. Culturing cells can result in cell growth, differentiation, and/or division.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

The term “exosomes” as used herein refers to small secreted vesicles (typically about 30 nm to about 250 nm (or largest dimension where the particle is not spheroid)) that may contain, or have present in their membrane or contained within their membrane, nucleic acid(s), protein, small molecule therapeutics, or other biomolecules and may serve as carriers of this cargo between diverse locations in a body or biological system. The term “exosomes” as used herein advantageously refers to extracellular vesicles that can have therapeutic properties, including, but not limited to LSC exosomes.

Exosomes may be isolated from a variety of biological sources including mammals such as mice, rats, guinea pigs, rabbits, dogs, cats, bovine, horses, goats, sheep, primates or humans. Exosomes can be isolated from biological fluids such as serum, plasma, whole blood, urine, saliva, breast milk, tears, sweat, joint fluid, cerebrospinal fluid, semen, vaginal fluid, ascetic fluid and amniotic fluid. Exosomes may also be isolated from experimental samples such as media taken from cultured cells (“conditioned media,” cell media, and cell culture media). Exosomes may also be isolated from tissue samples such as surgical samples, biopsy samples, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes may be isolated from freshly collected samples or from samples that have been stored frozen or refrigerated. Although not necessary, higher purity exosomes may be obtained if fluid samples are clarified before precipitation with a volume-excluding polymer, to remove any debris from the sample. Methods of clarification include centrifugation, ultracentrifugation, filtration or ultrafiltration.

The genetic information within the extracellular vesicle such as an exosome may easily be transmitted by fusing to the membranes of recipient cells, and releasing the genetic information into the cell intracellularly. Though exosomes as a general class of compounds represent great therapeutic potential, the general population of exosomes are a combination of several class of nucleic acids and proteins which have a constellation of biologic effects both advantageous and deleterious.

The term “vesicle” or “extracellular vesicle” as used herein can refers to a vesicle secreted by cells or derived from cells (e.g., via extrusion process) that may have a larger diameter than that referred to as an “exosome.” Vesicles and nanovesicles (alternatively named “microvesicle” or “membrane vesicle”) may have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.). Typically, at least part of the membrane of the extracellular vesicle is directly obtained from a cell (also known as a donor cell).

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

As used berein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, macaque, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. ENGINEERED EXTRACELLULAR VESICLES AND RELATED COMPOSITIONS

Embodiments of the present disclosure include compositions comprising a plurality of engineered extracellular vesicles (EVs). In accordance with these embodiments, the compositions include a plurality of EVs comprising (i) at least one membrane-associated protein on the surface of the plurality of EVs; and/or (ii) at least one therapeutic agent loaded into the plurality of EVs.

In some embodiments, the plurality of EVs are derived from a cell. In some embodiments, the plurality of EVs are derived from a cell selected from the group consisting of: HeLa cells, HEK293 cells, HEK293 derived cells, Vero cells, CHO cells, CHO-K1 cells, CHO-derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, and RK cells. In some embodiments, the plurality of EVs are derived from a lung spheroid cell (LSC).

In some embodiments, the plurality of EVs comprise liposomes. In some embodiments, the plurality of EVs comprise exosomes. As would be recognized by one of ordinary skill in the are based on the present disclosure, EVs include any membrane bound nanometer-scale vesicles comprising a lumen. Compositions of the present disclosure also include a plurality of EVs comprising more than one type of EV (e.g., exosomes and liposomes).

The size of the EVs will depend on the methods employed to derive them from, for example, a parent cell, as well as other factors, such as how the EV will be delivered or administered to a subject for a therapeutic purpose. In some embodiments, the plurality of EVs comprise an average size ranging from about 30 nm to about 1000 nm. In some embodiments, the plurality of EVs comprise an average size ranging from about 30 nm to about 900 nm. In some embodiments, the plurality of EVs comprise an average size ranging from about 30 nm to about 800 nm. In some embodiments, the plurality of EVs comprise an average size ranging from about 30 nm to about 700 nm. In some embodiments, the plurality of EVs comprise an average size ranging from about 30 nm to about 600 nm. In some embodiments, the plurality of EVs comprise an average size ranging from about 30 nm to about 500 nm. In some embodiments, the plurality of EVs comprise an average size ranging from about 30 nm to about 400 nm. In some embodiments, the plurality of EVs comprise an average size ranging from about 30 nm to about 300 nm. In some embodiments, the plurality of EVs comprise an average size ranging from about 30 nm to about 200 nm. In some embodiments, the plurality of EVs comprise an average size ranging from about 50 nm to about 1000 nm. In some embodiments, the plurality of EVs comprise an average size ranging from about 100 nm to about 1000 nm. In some embodiments, the plurality of EVs comprise an average size ranging from about 200 nm to about 1000 nm. In some embodiments, the plurality of EVs comprise an average size ranging from about 300 nm to about 1000 nm. In some embodiments, the plurality of EVs comprise an average size ranging from about 400 nm to about 1000 nm. In some embodiments, the plurality of EVs comprise an average size ranging from about 500 nm to about 1000 nm. In some embodiments, the plurality of EVs comprise an average size ranging from about 600 nm to about 1000 nm. In some embodiments, the plurality of EVs comprise an average size ranging from about 700 nm to about 1000 nm. In some embodiments, the plurality of EVs comprise an average size ranging from about 800 nm to about 1000 nm. In some embodiments, the plurality of EVs comprise an average size ranging from about 900 nm to about 1000 nm. In some embodiments, the plurality of EVs comprise an average size ranging from about 100 nm to about 900 nm. In some embodiments, the plurality of EVs comprise an average size ranging from about 200 nm to about 800 nm. In some embodiments, the plurality of EVs comprise an average size ranging from about 300 nm to about 700 nm. In some embodiments, the plurality of EVs comprise an average size ranging from about 300 nm to about 600 nm. In some embodiments, the plurality of EVs comprise an average size ranging from about 200 nm to about 500 nm. In some embodiments, the plurality of EVs comprise an average size ranging from about 50 nm to about 300 nm. In some embodiments, the plurality of EVs comprise an average size ranging from about 100 nm to about 200 nm. In some embodiments, the plurality of EVs comprise an average size ranging from about 50 nm to about 200 nm. In some embodiments, the plurality of EVs comprise an average size of about 100 nm. In some embodiments, the plurality of EVs comprise an average size of about 125 nm. In some embodiments, the plurality of EVs comprise an average size of about 150 nm. In some embodiments, the plurality of EVs comprise an average size of about 175 nm. In some embodiments, the plurality of EVs comprise an average size of about 200 nm.

Embodiments of the present disclosure include compositions comprising a plurality of engineered extracellular vesicles (EVs) comprising at least one membrane-associated protein on the surface of the plurality of EVs. In some embodiments, the at least one membrane-associated protein on the surface of the plurality of EVs comprises a viral-specific protein, or a derivative or fragment thereof. In some embodiments, the viral-specific protein comprises an antigenic epitope or derivative or fragment thereof capable of stimulating an immune response in a subject. In some embodiments, the viral-specific protein comprises a coronavirus Spike protein (S protein), or a derivative or fragment thereof. In some embodiments, the viral-specific protein comprises a receptor binding domain (RBD) of a coronavirus Spike protein (S protein), or a derivative or fragment thereof, capable of binding Angiotensin-converting enzyme 2 (ACE2).

As would be recognized by one of ordinary skill in the art based on the present disclosure, the at least one membrane-associated protein on the surface of the plurality of EVs can include other therapeutic agents. In some embodiments, the plurality of EVs of the present disclosure can be engineered to include peptide or protein-based therapeutic agents on their surfaces. In some embodiments, these therapeutic proteins can include therapeutic antibodies or derivatives or variants thereof. In some embodiments, the plurality of EVs of the present disclosure can be engineered to include polyclonal antibodies, monoclonal antibodies, Fv, single chain variable fragments (scFv), Fab and F(ab)₂ fragments, VHH fragments, diabodies, synthetic epitopes, single domain antibodies, human antibodies, and humanized antibodies (See, e.g., Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

Embodiments of the present disclosure include compositions comprising a plurality of engineered extracellular vesicles (EVs) comprising at least one therapeutic agent loaded into the plurality of EVs. In some embodiments, the at least one therapeutic agent loaded into the plurality of EVs comprises mRNA encoding an antigenic epitope or derivative or fragment thereof capable of stimulating an immune response in a subject. In some embodiments, the mRNA encodes an antigenic epitope or a derivative or fragment thereof capable of stimulating an immune response in a subject from a coronavirus Spike protein (S protein), or a derivative or fragment thereof. In some embodiments, the mRNA encodes an antigenic epitope or a derivative or fragment thereof capable of stimulating an immune response in a subject from a receptor binding domain (RBD) of a coronavirus Spike protein (S protein), or a derivative or fragment thereof, capable of binding Angiotensin-converting enzyme 2 (ACE2). In some embodiments, the at least one therapeutic agent loaded into the plurality of EVs comprises a plurality of different mRNAs encoding a plurality of different antigenic epitopes or derivatives or fragments thereof capable of stimulating an immune response in a subject.

In some embodiments, the plurality of EVs include cell surface proteins capable of binding a virus, such as a coronavirus. In some embodiments, the coronavirus is selected from the group consisting of 229E, NL63, OC43, HKU1, MERS-CoV, SARS-CoV, and SARS-CoV-2. Coronaviruses are a family of enveloped RNA viruses (positive-strand RNA viruses) that are distributed widely among mammals and birds, causing principally respiratory or enteric diseases but in some cases neurologic illness or hepatitis. Individual coronaviruses usually infect their hosts in a species-specific manner, and infections can be acute or persistent. Infections are transmitted mainly via respiratory and fecal-oral routes. The most distinctive feature of this viral family is genome size: coronaviruses have the largest genomes among all RNA viruses, including those RNA viruses with segmented genomes. This expansive coding capacity seems to both provide and necessitate a wealth of gene-expression strategies.

In some embodiments, the at least one therapeutic agent loaded into the plurality of EVs comprises at least one mRNA encoding a tumor-associated antigen (TAA). In some embodiments, the TAA is selected from the group consisting of: MAGE-C1, MAGE-C2, MAGE-C3, MAGE-A3, NY-SEO-1, survivin, 5 T4, MUC1, PSA, PSCA, PSMA, STEAP1, PAP, tyrosinase, GP100, and CT7, or any combination thereof.

In some embodiments, the at least one therapeutic agent loaded into the plurality of EVs comprises at least one mRNA encoding an immunostimulant. In some embodiments, the immunostimulant is selected from the group consisting of: IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, INF-gamma, GM CSF, G-CSF, M-CSF, LT-βor TNF-α, OX40L, CD40L, and CD7, or any combination thereof. In some embodiments, the immunostimulant is IL-12.

In some embodiments, the at least one therapeutic agent loaded into the plurality of EVs comprises at least one small molecule. In some embodiments, the at least one small molecule is an anti-cancer drug. In some embodiments, the anti-cancer drug is selected from the group consisting of: a kinase inhibitor, an ALK inhibitor, a c-Met inhibitor, an EGFR inhibitor, an FLT3 inhibitor, a VEGFR/FGFR/PDGFR inhibitor, a TRK inhibitor, Bcr-Abll inhibitor, a BTK inhibitor, a JAK inhibitor, a BRAF/MEK/ERK inhibitor, a CDK inhibitor, a PI3K/AKT/mTOR inhibitor, an EZH2 inhibitor, an HDAC inhibitor, an IDH1/2 inhibitor, and a BCL-2 inhibitor, or any combinations thereof. In some embodiments, the anti-cancer drug is a VEGFR/FGFR/PDGFR inhibitor selected from the group consisting of: nintedanib, sorafenib, sunitinib, lenvatinib, pazopanib, axitinib, cabozantinib, tivozanib, apatinib, anlotinib, fruquintinib, erdafitinib, pemigatinib, avapritinib, imatinib, regorafenib, ripretinib, cediranib, dovitinib, motesanib, crenolanib, lucitanib, vactosertib, vandetanib, selpercatinib, pralsetinib, sulfatinib, and brivanib.

In some embodiments, loading of the at least one therapeutic agent comprises encapsulating the at least one therapeutic agent in the EV membrane and/or encapsulating the at least one therapeutic agent within the lumen of the EV. As would be recognized by one of ordinary skill in the art based on the present disclosure, the at least one therapeutic agent loaded into the plurality of EVs can comprise combinations of mRNAs encoding a TAA, immunostimulants, or any other therapeutic mRNAs. In some embodiments, the at least one therapeutic agent loaded into the plurality of EVs comprises a gene editing agent (or nucleic acid molecule that encodes a gene editing agent), or an agent that is a component of a gene editing composition. In some embodiments, the gene editing agent includes, but is not limited to, circular RNA, circular DNA, ssRNA, ssDNA, and siRNA. In some embodiments, the gene editing agent is a component (or is a nucleic acid molecule that encodes a gene editing component) of a gene editing system, including but not limited to, a CRISPR-Cas system, a transcription activator-like effector nuclease (TALEN) system, a zinc-finger nuclease (ZFN) system, or a homing endonuclease or meganuclease based system.

In accordance with the above, embodiments of the present disclosure include compositions comprising a plurality of engineered extracellular vesicles (EVs) that comprise at least one membrane-associated protein on the surface of the plurality of EVs and at least one therapeutic agent loaded into the plurality of EVs. As described herein, embodiments of the present disclosure include compositions comprising a plurality of engineered EVs that comprise any combination of membrane-associated proteins described herein, as well as any combination of therapeutic agents described herein. In some embodiments, the plurality of EVs can have cargo that includes at least one therapeutic protein, peptide, polypeptide, nucleic acid molecule, polynucleotide, mRNA, siRNA, miRNA, antisense oligonucleotide, drug, or therapeutic small molecule. In some embodiments, the cargo can enhance binding to a virus and/or enhance a therapeutic effect that the EVs exert against a virus.

In accordance with the above embodiments, the present disclosure also includes a method of preventing and/or treating a viral infection comprising administering any of the compositions described herein to a subject. In some embodiments of the method, the virus is a coronavirus. In some embodiments, the coronavirus is selected from the group consisting of 229E, NL63, OC43, HKU1, MERS-CoV, SARS-CoV, and SARS-CoV-2, or any variants thereof.

In accordance with the above embodiments, the present disclosure also includes a method of treating cancer comprising administering any of the compositions described herein to a subject. In some embodiments of the method, the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration. In some embodiments of the method, the composition is administered via nebulization to lung tissue.

In some embodiments, the composition further comprises at least one pharmaceutically acceptable excipient or carrier. A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the EVs, a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered EVs and any corresponding cargo (e.g., vaccine). Adjusting the dose to achieve maximal efficacy based on the methods described above and other methods as are well-known in the art is well within the capabilities of the ordinarily skilled person in the art.

Embodiments of the present disclosure also includes methods of generating a plurality of EVs for the treatment and/or prevention of a disease. In accordance with these embodiments, the methods include culturing a plurality of parental cells from which the EVs are derived, such as lung spheroid cells (LSCs). Parental cells can be cultured in 2D or 3D cell culture platforms. In some embodiments, the method includes subjecting the plurality of parental cells to an extrusion process to produce the plurality of EVs having the desired characteristics. In some embodiments, the extrusion process comprises passing the parental cells (e.g., LSCs) through an extruder comprising at least one of a 5 μm, a 1 μm, and/or a 400 nm pore-sized membrane filters. As would be recognized by one of ordinary skill in the art, other filter sizes and combinations can be used in the extrusion process, depending on the EV size and characteristics desired. In some embodiments, the method further includes purifying and concentrating the plurality of EVs using ultrafiltration or other filtration means known in the art. In some embodiments, the EVs can be selected, sorted, purified, or concentrated based on the use of one or more cell surface proteins.

Embodiments of the present disclosure also include compositions that include a plurality of exosomes derived from a cell. In general, the exosomes can be derived from any cell, including but not limited to a lung spheroid cell (LSC), according to the methods described further herein, as well as those methods described in PCT/US2019/039721, which is herein incorporated by reference in its entirety. As would be recognized by one of ordinary skill in the art based on the present disclosure, exosomes derived from cells are not naturally-occurring; however, they may share one or more features of the parent cell from which they were derived. In some embodiments, the plurality of exosomes are derived from a cell. In some embodiments, the plurality of exosomes are derived from a cell selected from the group consisting of: Hela cells, HEK293 cells, HEK293 derived cells, Vero cells, CHO cells, CHO-K1 cells, CHO-derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, and RK cells. In some embodiments, the plurality of exosomes are derived from a lung spheroid cell (LSC).

In accordance with these embodiments, the compositions of the present disclosure include a plurality of exosomes comprising at least one membrane-associated protein on the surface of the plurality of exosomes (e.g., a cell surface receptor or binding protein). In some embodiments, the membrane-associated protein on the surface of the plurality of exosomes is a viral-specific protein, such as a viral protein, peptide, or polypeptide that can induce an immunogenic response in a subject (e.g., a viral antigen or epitope). In some embodiments, the membrane-associated protein on the surface of the exosomes comprises TAA capable of inducing an immune response in a subject. In some embodiments, the plurality of exosomes can be generated to include one or more therapeutic agents contained within their membranes (e.g., cargo), which can further enhance an immune response in a subject. Such therapeutic agents can include any protein, peptide, polypeptide, nucleic acid, small molecule compound, or any combinations or derivatives thereof that can enhance an immune response in a subject.

In accordance with the above embodiments, the compositions of the present disclosure can be formulated as a pharmaceutically acceptable composition for administering to a subject in need thereof to treat and/or prevent a disease or condition. In some embodiments, the compositions of the present disclosure are stable at room temperature (e.g., 15-25° C.). In some embodiments, the compositions of the present disclosure are stable below room temperature. In some embodiments, the compositions of the present disclosure are stable above room temperature. In some embodiments, the compositions of the present disclosure are stable at room temperature for at least 6 hours. In some embodiments, the compositions of the present disclosure are stable at room temperature for up to an including 6 months. In some embodiments, the compositions of the present disclosure are stable at room temperature from about 1 day to about 6 months, from about 1 day to about 5 months, from about 1 day to about 4 months, from about 1 day to about 3 months, from about 1 day to about 2 months, from about 1 day to about 1 month, from about 1 day to about 4 weeks, from about 1 day to about 3 weeks, from about 1 day to about 2 weeks, and from about 1 day to about 1 week.

In some embodiments, the compositions of the present disclosure can be formulated as a composition that comprises a pharmaceutically acceptable excipient and/or carrier or diagnostically acceptable excipient and/or carrier, including but not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the EVs, a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered EVs and any corresponding cargo. Adjusting the dose to achieve maximal efficacy based on the methods described above and other methods as are well-known in the art is well within the capabilities of the ordinarily skilled person in the art.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The pharmaceutically acceptable compositions described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, inhalable, topical, injectable, immediate-release, pulsatile-release, delayed-release, or sustained release). The pharmaceutically acceptable compositions may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20.

### 3. THERAPEUTIC METHODS

Embodiments of the present disclosure also include a method of treating a viral infection comprising administering any of the compositions described above to a subject in need thereof. In some embodiments, the composition is administered orally, parenterally, intramuscularly, intraperitoneally, intravenously, intracerebroventricularly, intracistemally, subcutaneously, via injection or infusion, via inhalation, spray, nasal, vaginal, rectal, sublingual, or topical administration. In some embodiments, the composition is administered via nebulization to lung tissue.

In some embodiments, administration of the plurality of EVs or exosomes reduces viral load in the subject. As would be recognized by one of ordinary skill in the art based on the present disclosure, pharmaceutical compositions comprising a plurality of EVs or exosomes can be administered in an amount effective such that a desired therapeutic result is achieved (e.g., immunogenic response). In some embodiments, the composition is administered (e.g., via inhalation) at a dose of about 1×10⁷ to about 1×10¹³ particles per kg of body weight. In some embodiments, the composition is administered at a dose of about 1×10⁸ to about 1×10¹² particles per kg of body weight. In some embodiments, the composition is administered at a dose of about 1×10⁹ to about 1×10¹¹ particles per kg of body weight. In some embodiments, the composition is administered at a dose of about 1×10⁷ particles per kg of body weight, about 1×10⁸ particles per kg of body weight, about 1×10⁹ particles per kg of body weight, about 1×10¹⁰ particles per kg of body weight, about 1×10¹¹ particles per kg of body weight, about 1×10¹² particles per kg of body weight, about 1×10¹³ particles per kg of body weight, about 1×10¹⁴ particles per kg of body weight, or about 1×10¹⁵ particles per kg of body weight.

In accordance with these embodiments, the plurality of EVs or exosomes of the present disclosure can persist in the subject's tissues (e.g., lung tissue) for at least 72 hours after administration. In some embodiments, the plurality of EVs or exosomes persist in a subject for at least 12 hours, at least 24 hours, at least 36 hours, at least 48 hours, at least 60 hours, at least 72 hours, at least 84 hours, and at least 96 hours. In some embodiments, the plurality of EVs or exosomes are administered every 24 hours, every 48 hours, every 72 hours, or every 96 hours, depending on the dose being administered and the subject's physiological characteristics.

In some embodiments, a single dose of the plurality of EVs or exosomes of the present disclosure can exert a beneficial effect (e.g., induce an immunogenic response) on a subject. In some embodiments, two or more doses are required to provide a beneficial effect. In some embodiments, three or more doses are required to provide a beneficial effect. In some embodiments, four or more doses are required to provide a beneficial effect. In some embodiments, five or more doses are required to provide a beneficial effect. In some embodiments, six or more doses are required to provide a beneficial effect. In some embodiments, seven or more doses are required to provide a beneficial effect. In some embodiments, eight or more doses are required to provide a beneficial effect. In some embodiments, nine or more doses are required to provide a beneficial effect. In some embodiments, ten or more doses are required to provide a beneficial effect.

In some embodiments, the present disclosure encompasses methods of treating a pathological condition of a human subject, wherein the method comprises administering to a region of the respiratory tract of the human subject a pharmaceutical composition comprising a plurality of EVs in an amount effective in modulating a pathological condition when delivered to the human subject in need thereof. In some embodiments, the pathological condition is a viral infection (e.g., COVID-19), an immune disorder, or cancer. The various compositions of the present disclosure provide dosage forms, formulations, and methods that confer advantages and/or beneficial pharmacokinetic profiles. A composition of the disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the disclosure present in an amount sufficient to provide the required concentration or dose of the composition to an environment of use to treat a disease disclosed herein.

A subject may be treated with a composition of the present disclosure or composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A composition or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously. A beneficial pharmacokinetic profile can be obtained by the administration of a formulation or dosage form suitable for once, twice, or three times a day administration in an amount sufficient to provide a required dose of the composition. Certain dosage forms and formulations may minimize the variation between peak and trough plasma and/or brain levels of compositions of the disclosure and in particular provide a sustained therapeutically effective amount of the compositions. The present disclosure also contemplates a formulation or dosage form comprising amounts of one or more composition of the disclosure that results in therapeutically effective amounts of the composition over a dosing period, in particular a 24 h dosing period. A medicament or treatment of the disclosure may comprise a unit dosage of at least one composition of the disclosure to provide therapeutic effects. A “unit dosage or “dosage unit” refers to a unitary (e.g., a single dose), which is capable of being administered to a subject, and which may be readily handled and packed, remaining as a physically and chemically stable unit dose comprising either the active agents as such or a mixture with one or more solid or liquid pharmaceutical excipients, carriers, or vehicles.

### 4. MATERIALS AND METHODS

Cell culture. Human LSCs were generated from healthy whole lung samples from the Cystic Fibrosis and Pulmonary Diseases Research and Treatment Center at the University of North Carolina at Chapel Hill and expanded as previously described. LSCs were plated on a fibronectin-coated (Coming Incorporated, Coming, NY, USA) flask and maintained in Iscove's Modified Dulbecco's Media (IMDM; ThermoFisher Scientific, Waltham, MA, USA) containing 20% fetal bovine serum (FBS; Coming Incorporated, Coming, NY, USA), 1% L-glutamine (ThermoFisher Scientific, Waltham, MA, USA), 0.5% Gentamicin (ThermoFisher Scientific, Waltham, MA, USA), and 0.18% 2-mercaptoethanol (ThermoFisher Scientific, Waltham, MA, USA). Human embryonic kidney (HEK) 293T cells were purchased from American Type Culture Collection (ATCC; American Type Culture Collection, Manassas, VA, USA). HEK cells were plated on a flask and maintained in Minimum Essential Media (MEM; ThermoFisher Scientific, Waltham, MA, USA) containing 10% FBS, 1% L-glutamine, 0.5% Gentamicin, and 0.18% 2-mercaptoethanol. Human bronchial epithelial cells were purchased from Lonza (CC-2540B; Lonza, Basel, Switzerland) and maintained according to manufacturer's instructions. Media changes on all cultures were performed every other day. LSCs and HEK cells were allowed to reach 70-80% confluence before generating serum-free secretome (Lung-Secretome, HEK-Secretome) as previously described. Lung- and HEK-Secretome were collected and filtered through a 0.22 μm filter to remove cellular debris. All procedures performed in this study involving human samples were in accordance with the ethical standard of the institutional research committee and with the guidelines set by the Declaration of Helsinki.

Exosome isolation and characterization. Lung-Exo and HEK-Exo were collected and isolated from Lung-Secretome and HEK-Secretome using an ultrafiltration method. Filtered secretome was pipetted into a 100 kDa Amicon centrifugal filter unit (MilliporeSigma, Burlington, MA, USA) and centrifuged at 400 RCF and 10° C. After all media passed through the centrifugal filter unit, remaining exosomes were detached from the filter and resuspended using 1× Dulbecco's phosphate-buffered saline (DPBS; ThermoFisher Scientific, Waltham, MA, USA) with 25 mM Trehalose (MilliporeSigma, Burlington, MA, USA) for further analysis. Pegylated Remote Loadable Liposomes (Lipo) were purchased from Avanti Polar Lipids (Avanti Polar Lipids, Inc, Alabaster, AL, USA). LSC-Exo, HEK-Exo, and Lipo were fixed with 4% paraformaldehyde (PFA; Electron Microscopy Sciences, Hatfield, PA, USA) and 1% glutaraldehyde (Sigma-Aldrich, St. Louis, MO, USA) onto 100 mesh copper grids (Electron Microscopy Sciences, Hartfield, PA, USA) for transmission electron microscopy imaging (JEOL JEM-2000FX, Peabody, MA, USA). Samples were stained with Vanadium Negative Stain (ab172780; Abcam, Cambridge, United Kingdom). Sample concentrations and mean diameters were quantified by nanoparticle tracking analysis before and after fluorescent label loading (NanoSight NS3000, Malvem Panalytical, Malvem, UK).

Nanoparticle fluorescent label loading. RFP (ab268535; Abcam, Cambridge, United Kingdom) was loaded into Lung-Exo and Lipo particles via electroporation, yielding RFP-Exo and RFP-Lipo. 1 billion nanoparticles from each sample were diluted in Gene Pulser® Electroporation Buffer (Bio-Rad, Hercules, CA, USA) at a 1:9 ratio of nanoparticles to buffer. 10 μg of RFP were added to the nanoparticle-buffer solution and transferred to an ice-cold 0.4 cm Gene Pulser/MicroPulser Electroporation Cuvette (Bio-Rad, Hercules, CA, USA). The electroporation cuvette was inserted into the Gene Pulser Xcell™ Total System (Bio-Rad, Hercules, CA, USA) and electroporated under the following conditions: pulse type: square waveforms; voltage: 200V; pulse length: 10 msec; number of pulses: 5; pulse interval: 1 sec. Electroporation buffer was filtered out of the fluorescently labeled nanoparticles by the ultrafiltration method described above. Lung-Exo, HEK-Exo, and Lipo were incubated with DiD labeling solution (V22889; ThermoFisher Scientific, Waltham, MA, USA) according to manufacturer's instructions. 10 μg of GFP-encoding mRNA DasherGFP (Aldevron, Fargo, ND, USA) and 10 μg of RFP were loaded into LSC-Exo, HEK-Exo, and Lipo nanoparticles via the electroporation method described above.

Nanoparticle spike protein loading. Full length DNA sequence of the S surface glycoprotein (SARS-CoV 2 isolate Wuhan-Hu-1, Gene ID: 43740568, NC_045512.2:21563-25384) was used to design a plasmid for in-vitro transcription (IVT). Briefly, a stretch of DNA sequence with two restriction sites, I-CeuI and I-SceI, was cloned into pCR™4Blunt-TOPO vector (45-0031; ThermoFisher Scientific, Waltham, MA, USA). The full length of the S-protein sequence was split to design two gBlocks (Integrated DNA Technologies, Coralville, Iowa, USA), each of which is approximately 1.5 kb and 2.3 kb long. The T7 promoter sequence (5′-taatacgactcactataggg-3′) was added to the 5′ end of 1.5 kb gBlock. The two gBlocks were ligated into the backbone vector using I-CeuI, BstEII, and I-SceI. Following sequence confirmation by Sanger sequencing (Genewiz, South Plainfield, NJ, USA), the full-length S-protein DNA sequence, cut by I-CeuI and I-SceI, was extracted from TAE agarose gel. IVT was done following the manufacturer's manual of the MEGAscript® Kit (AM1333; ThermoFisher Scientific, Waltham, MA, USA). High yield RNA was treated with DNase and cleaned up using the Monarch® RNA cleanup kit (T2040L; New England Biolab, Ipswich, MA, USA). Size-confirmed pure IVT S-protein mRNA was aliquoted and stored in −80° C. until nanoparticle loading. S-protein mRNA was loaded into LSC-Exo and Lipo nanoparticles via the electroporation method described above.

SDS-PAGE and western blot. Native and fluorescently-labeled LSC-Exo, HEK-Exo, and Lipo were further characterized by immunoblotting. Samples were lysed, denatured, and reduced by Laemmli sample buffer (Bio-Rad, Hercules, CA, USA) and β-mercaptoethanol (Bio-Rad, Hercules, CA, USA) at 90° C. for 5 minutes. Protein samples and molecular ladder (Precision Plus Protein Unstained Standards; Bio-Rad, Hercules, CA, USA) were loaded into a 10% acrylamide precast Tris-Glycine gel (Bio-Rad, Hercules, CA, USA) for sodium dodecyl sulfate polyacrylamide gel electrophoresis (SDS-PAGE) separation. Gels were run at a stacking voltage of 100V until samples ran out of the wells, followed by a constant voltage of 200V. Gels were visualized and imaged in a Bio-Rad Imager (Bio-Rad, Hercules, CA, USA). Gels were transferred onto polyvinylidene fluoride membranes (PVDF; Bio-Rad, Hercules, CA, USA) using the Bio-Rad wet electroblotting transfer system (Bio-Rad, Hercules, CA, USA). Following transfer, membranes were washed three times in 1× phosphate-buffered saline with 0.1% Tween detergent (PBS-T; MilliporeSigma, Burlington, MA, USA) for 5 minutes each and blocked using 5% milk in PBS-T for one hour at room temperature. Membranes were blotted against anti-β-Actin (ab6276; Abcam, Cambridge, United Kingdom), anti-CD63 (PAS-100713; ThermoFisher Scientific, Waltham, MA, USA), anti-GFP (ab290; Abcam, Cambridge, United Kingdom), and anti-RFP (ab62341; Abcam, Cambridge, United Kingdom) primary antibodies in 5% milk in PBS-T and incubated at 4° C. for one week. After incubation, membranes were incubated with the corresponding goat anti-rabbit (ab6721; Abcam, Cambridge, United Kingdom) and goat anti-mouse (ab6789; Abcam, Cambridge, United Kingdom) HRP-conjugated secondary antibodies for 1 hour at room temperature. Membranes were then visualized using Clarity Western ECL Substrate (Bio-Rad, Hercules, CA, USA) and imaged in a Bio-Rad Imager (Bio-Rad, Hercules, CA, USA). Band intensities were analyzed using ImageJ analysis software (NIH; imagej.nih.gov/ij/).

Tissue clearing and imaging. Mouse lungs were cleared using the BoneClear protocol. Anesthetized mice were perfused with DPBS and 50 μg/mL heparin (Sigma-Aldrich, St. Louis, MO, USA). Lung tissues were dissected and fixed in a DPBS, 0.5% PFA (Electron Microscopy Sciences, Hatfield, PA, USA), and 10% sucrose (Sigma-Aldrich, St. Louis, MO, USA) solution at room temperature, then further fixed ovemight in a DPBS and 0.5% PFA solution at 4° C. Fixed lung samples were incubated in a methanol (VWR, Randor, PA, USA) gradient of 20%, 40%, 60%, 80%, and 100% for two hours per percentage at room temperature. Then, the samples were decolorized overnight in a 30% hydrogen peroxide (Sigma-Aldrich, St. Louis, MO, USA) and 100% methanol solution at a 1:10 ratio respectively at 4° C. Lung tissues were then incubated in a reverse methanol gradient at room temperature and permeabilized with a DPBS, 0.02% Triton X-100 (Sigma-Aldrich, St. Louis, MO, USA), 0.01% sodium deoxycholate (Sigma-Aldrich, St. Louis, MO, USA), 10% dimethyl sulfoxide (DMSO; Sigma-Aldrich, St. Louis, MO, USA), 25 mM EDTA (Sigma-Aldrich, St. Louis, MO, USA) solution at 37° C. overnight. Lung samples were then blocked with a DPBS, 0.02% Triton X-100, 10% DMSO, 5% normal donkey serum (Sigma-Aldrich, St. Louis, MO, USA), and 25 mM EDTA solution overnight at 37° C. Lung tissues were immunolabeled with anti-RFP (ab62341; Abcam, Cambridge, United Kingdom) primary antibody diluted in a DPBS, 0.02% Tween-20, 1 g/mL heparin, 5% normal donkey serum, and 25 mM EDTA solution at a 1:200 ratio respectively for one weck at 37° C. Tissues were washed and further immunolabeled with Cy3 (711-165-152; Jackson ImmunoResearch Laboratories, West Grove, PA, USA) secondary antibody diluted in a DBPS, 0.02% Tween-20, 1 g/mL heparin, 5% normal donkey serum, and 25 mM EDTA solution at a 1:500 ratio respectively for 5 days at 37° C. Lung tissues were washed, incubated in a methanol gradient, then incubated twice in a dichloromethane (Sigma-Aldrich, St. Louis, MO, USA) and methanol solution at a 1:2 ratio respectively at room temperature, and followed by four incubations in 100% dichloromethane. Finally, lung samples were cleared three times with 100% dibenzyl-ether (Sigma-Aldrich, St. Louis, MO, USA) at room temperature and imaged using a custom-built light sheet microscope. Whole lung images and movies were captures using Imaris image analysis software (Imaris, Oxford Instruments, Abingdon, United Kingdom). (These data can be made available upon request.)

Particle segmentation. Cleared mouse lungs were analyzed using ImageJ analysis software. In each image, pixels that belonged to exosomes or liposomes were segmented via thresholding, during which the intensity threshold was decided manually. The selection brush tool was used to refine the masks generated by thresholding and to segment the airway regions. Quantification of areas or pixels was then performed based on the extracted masks.

Air-liquid interface system. A model of the human airway at the air-liquid interface was created by seeding human bronchial epithelial cells onto a 0.4 μm pore polycarbonate membrane and lung parenchymal cells onto a 6.5 mm well in a transwell system (Coming Incorporated, Coming, NY, USA). Cells were maintained for one week before administering DiD-labeled exosomes and liposomes to the human bronchial epithelial cells. Nuclei in the transwell wells were visualized by adding NucBlue™ Live ReadyProbes™ Reagent (R37605; ThermoFisher Scientific, Waltham, MA, USA) to the media and analyzed using ImageJ analysis software.

DPI fabrication. A DPI for nanoparticle inhalation to mice was fabricated as previously described, with modifications. The inhalation apparatus was adapted by using a plastic microcentrifuge tube as the powder receptacle. A plastic 250 mL centrifuge tube was attached to the powder receptacle to serve as a containment chamber for the un-anesthetized mouse; this optimized mouse muzzle orientation. A DPI for nanoparticle inhalation to primates was assembled using the RS01 high-resistance DPI (239700002AA; Berry Global, Evansville, IN, USA) connected to an aerosol chamber inhaler spacer (Canack Technology Ltd., Vancouver, Canada).

Animal procedures. Seven-week-old male CD1 mice (022) were obtained from Charles River Laboratory (Wilmington, MA, USA). RFP-Exo, RFP-Lipo, Lung-Exo, HEK-Exo, and Lipo were administered via jet nebulization (Pari Trek S Portable 459 Compressor Nebulizer Aerosol System, PARI, Stamberg, Germany) or dry-powder inhalation as described above. Fluorescently-labeled nanoparticles were given in a single dose of 10⁹ particles per kg of body weight. Immediately after sacrifice, the lungs, heart, liver, kidneys, spleen, cecum, and brain were excised and imaged using an Xenogen Live Imager (PerkinElmer, Waltham, MA, USA). Blood was collected in Vacuette ethylenediaminetetraacetic acid (EDTA) tubes (Greiner Bio-One, Kremsmünster, Austria) and centrifuged at maximum speed for 5 minutes to separate out serum. All animal studies complied with the requirements of the Institutional Animal Care and Use Committee (IACUC) of North Carolina State University.

IgG and SIgA antibody titers. To collect BALF, the trachea was exposed by thoracotomy and a transverse incision was made at the top of the bronchial bifurcation. A needle was inserted into the trachea to wash the lungs with 200 μL of DPBS. Washing was repeated three times for a total of 600 μL wash fluid. To collect NPLF, the trachea was cut in the middle and the nasopharynx was washed upwards from the incision with 200 μL DBPS. Washing was repeated three times for a total of 600 μL wash fluid. Spike protein-specific IgG (20154; Cell Signaling Technology, Danvers, MA, USA) and SIgA (58873; Cell Signaling Technology, Danvers, MA, USA) from BALF and NPLF were measured by ELISA per manufacturer's instructions.

Nonhuman primate studies. Three chlorocebus sabaeus monkeys were housed at Bioqual. The primates received a single dose of 109 lyophilized fluorescently-labeled Lung-Exo particles per kg of body weight via DPI. The primates were necropsied 24 hours and 1 week after dry powder inhalation of Lung-Exo. All animal studies complied with the requirements under local, state, and federal regulations and were approved by the Bioqual IACUC.

Histology. Immunostaining was performed on transwell membranes and tissue slides fixed in 4% PFA (Electron Microscopy Sciences, Hatfield, PA, USA) in DPBS for 30 minutes, followed by permeabilization and blocking with Dako Protein blocking solution (Aglient Technologies, Santa Clara, CA, USA) with 0.1% saponin (Sigma-Aldrich, St. Louis, MO, USA) at room temperature for 1 hour. Membranes were immunolabeled with anti-MUC5b (ab77995; Abcam, Cambridge, United Kingdom) primary antibody diluted in Dako Protein blocking solution and its corresponding goat anti-mouse (A10667; Invitrogen, Waltham, MA, USA) AF488-conjugated secondary antibody diluted in Dako Protein blocking solution. Membranes and slides were mounted with ProLong Gold Antifade Mountant (Invitrogen, Waltham, MA, USA) and ProLong Gold Antifade Mountant with DAPI (Invitrogen, Waltham, MA, USA). Membrane and slides were imaged on the Olympus FLUOVIEW CLSM (Olympus; FV3000, Shinjuku, Tokyo, Japan) with an Olympus UPlanSAPO 10× objective (Olympus; 1-U2B824, Shinjuku, Tokyo, Japan) and Olympus UPlanSAPO 60× objective (Olympus; 1-U2B832, Shinjuku, Tokyo, Japan). H&E staining was performed on tissue slides (Hematoxylin HHS16; Eosin 318906; Sigma-Aldrich, St. Louis, MO, USA) and imaged on the Leica DMi8 (Leica Microsystems, Wetzlar, Germany). Tissue slides were analyzed using ImageJ analysis software.

AFM imaging. Lung-Exo, HEK-Exo, and Lipo were prepared as freshly isolated particles in PBS and Trehalose solution (Fresh), lyophilized powder (Lyophilized), and lyophilized powder reconstituted in DNase/RNase-free distilled water (Invitrogen, Waltham, MA, USA) (Reconstituted). Samples were added onto coverslips functionalized by (3-Aminopropyl)triethoxysilane (APTMS; Sigma-Aldrich, St. Louis, MO, USA) as previously described. Samples were imaged on the MFP-3D AFM (Asylum Research, Oxford Instruments, Abingdon, United Kingdom) with a silicon scanning probe microscopy-sensor probe (ARROW-NCR-50; Neuchâtel, Switzerland). Height and diameter measurements were analyzed using Asylum Research Software Version 16 (Asylum Research, Oxford Instruments, Abingdon, United Kingdom) ran on Igor Pro 6 (WaveMetrics, Tigard, OR USA).

Particle counting. Lyophilized Lung-Exo, HEK-Exo, and Lipo were encapsulated in Quali V Inhalation capsules (Qualicaps, Inc., Whitsett, NC, USA) and inserted into low-, medium-, high-, and ultra-high-resistance DPIs (Berry Global, Evansville, IN, USA). The DPIs were inserted into a negative-pressure vacuum chamber to aerosolize the samples. Samples were aerosolized for 30 seconds, followed by particle size distribution measurement using the TSI AeroTrak particle counter (9306-V2, TSI, Shoreview, MN, USA) for 1-minute time intervals at ambient room temperature.

Statistical analysis. Statistical analysis was performed using GraphPad Prism analysis software (GraphPad Software Inc., San Diego, CA, USA). Results are shown as the mean±standard deviation. Comparisons among twos groups were performed using an unpaired t-test, followed by Welch's correction test. Comparisons among more than two groups were performed using a parametric one-way ANOVA test, followed by Bonferroni's multiple comparisons test. p≤0.05 was considered statistically significant. The legend is as follows: *p-values≤0.05; **p-values≤0.01; ***p-values≤0.001; ****p-values≤0.0001.

Flow cytometry of bead-bound exosomes. For staining of ACE2 receptor on exosome surfaces, 5×10⁹ exosomes were suspended in 50 μL of DPBS and incubated with 50 μL of 4 μm aldehyde/sulfate latex beads (10⁶) for 15 mins at room temperature (RT) and then moved to 4° C. overnight. 100 μL of 200 mM glycine buffer was added to the above solution and incubated for 30 mins to stop the binding of exosomes with beads. After centrifugation and washing, the pellet was blocked with 100 μL of 5% BSA and then stained with ACE2 antibodies (PA5-85139, Invitrogen) for 1 h at RT. After three washes with MACS flow buffer, the bead-bound-exosomes were resuspended with flow buffer with anti-rabbit IgG with Alexa Fluor® 647 (ab150083, Abcam) for 1 h at 4° C. After that, bead-bound-exosomes were washed three times for subsequent flow cytometry assay, which were conducted with a CytoFLEX flow cytometer (Beckman Coulter, Brea, CA, USA). FCS Express V6 software was used to analyze flow cytometry data.

SARS-CoV-2 pseudovirus neutralization assay in vitro. SARS-CoV-2pseudovirus carrying the GFP reporter (C1110G) was purchased from Montana Molecular. LSC-Exo, HEK-Exo, or rhACE2 at the indicated concentrations were incubated with SARS-CoV-2 pseudovirus for 30 mins at 37° C. After incubation, the mixture was added to A549 cells expressing ACE2 and incubated for another 24 h. The GFP signals from infected cells were detected by fluorescence multi-mode microplate (Infinite M Plex, Tecan Inc.). Additionally, the percentage of infected A549 cells was quantified by flow cytometry assay.

Mouse studies using SARS-CoV-2 pseudovirus, SARS-CoV-2 D614G pseudovirus carrying the GFP reporter (C1120G) was purchased from Montana Molecular. SARS-CoV-2 Delta pseudovirus was constructed by co-transfecting HEK293T cells with the plasmids of plv-spike-v8 (InvivoGen), pLenti-EF1pluciferase-PGK-RFP-T2A-PURO lentiviral reporter (LR252, ALSTEM), and pspax2 (64586, Addgene) via Lipofectamine 3000 (L3000015, ThermoFisher Scientific). After 48 to 72 hours, Delta pseudovirus was harvested from the culture medium through centrifugation (3000 rpm, 10 mins), aliquoted, and stored at −80° C. until used.

All mice studies complied with the requirements of the Institutional Animal Care and Use Committee (IACUC) at North Carolina State University (protocol #19-806-B). Seven-eight weeks old female CD1 mice (Crl:CD1(ICR)) were purchased from Charles River Laboratory (Wilmington, MA, USA). LSC-Exo (10¹⁰ per kg of mouse weight), HEK-Exo (10¹⁰ per kg of mouse weight) or rhACE2 (30 μg per kg of mouse weight) were administered via nebulization. After 2 hours, each mouse was challenged with 8×10⁸ GC of SARS-oV-2 pseudovirus or D614G pseudovirus or Delta pseudovirus. Lungs were excised and imaged at 24 hours post-challenge with an Xenogen Live Imager and then cryosectioned for exploring the distribution of SARS-CoV-2 pseudovirus in mouse lung.

Hamster studies with live SARS-CoV-2. Ten male and female Syrian golden hamsters (Envigo), 6-8 weeks old, were randomly divided into two groups. All hamsters were housed at Bioqual Inc. Hamsters were administered with PBS or LSC-Exo by nebulization (n=5 per group, 3F/2M). 2 hours after inhalation, the hamsters were challenged with 1.99×10⁴50% tissue culture infective dose (TCID₅₀) of SARS-CoV-2 using the intranasal and intratracheal routes (50 μL in each nare). Bronchoalveolar lavage (BAL), oral swabs (OS), and blood were collected at the indicated time. Hamsters were necropsied at day 7 post-challenge. All hamster studies were performed in compliance with all relevant local, state, and federal regulations and were approved by the Bioqual Institutional Animal Care and Use Committee (IACUC, 20-091P).

RNAscope in situ hybridization in hamsters. SARS-CoV-2 anti-sense-specific probe v-nCoV2019-S (ACD Cat. No. 848561) was purchased to target the positive-sense of the Spike sequence, and SARS-CoV-2 v-nCoV2019-S-sense (ACD Cat. No. 845701) was purchased to target the negative-antisense of the Spike sequence. Prior to performing RNAscope assay, slides were first deparaffinized in xylene, rehydrated, and incubated with RNAscope® H₂O₂ (ACD Cat. No. 322335) for 10 mins at room temperature, followed by treatment with retrieval in ACD P2 retrieval buffer (ACD Cat. No. 322000) for 15 mins at 98° C. After that, slides were incubated with protease plus (ACD Cat. No. 322331) for 30 min at 40° C. Probe hybridization and detection were performed through the RNAscope® 2.5 HD Detection Reagents-RED (ACD Cat. No.322360) according to the instructions of the manufacturer.

Statistical analysis. All quantitative experiments were conducted in triplicate independently. Data were shown as means±standard deviation. Student's two-tailed, unpaired t-test was used to analyze differences between any two groups. Comparisons of more than two groups were determined using one-way ANOVA followed by the post hoc Bonferroni test. Grouped data were determined by two-way ANOVA followed by Tukey post hoc test for multiple comparisons. P<0.05 was considered statistically significant.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to berein are hereby incorporated by reference in their entireties.

The present disclosure has multiple aspects, illustrated by the following non-limiting examples.

### Example 1

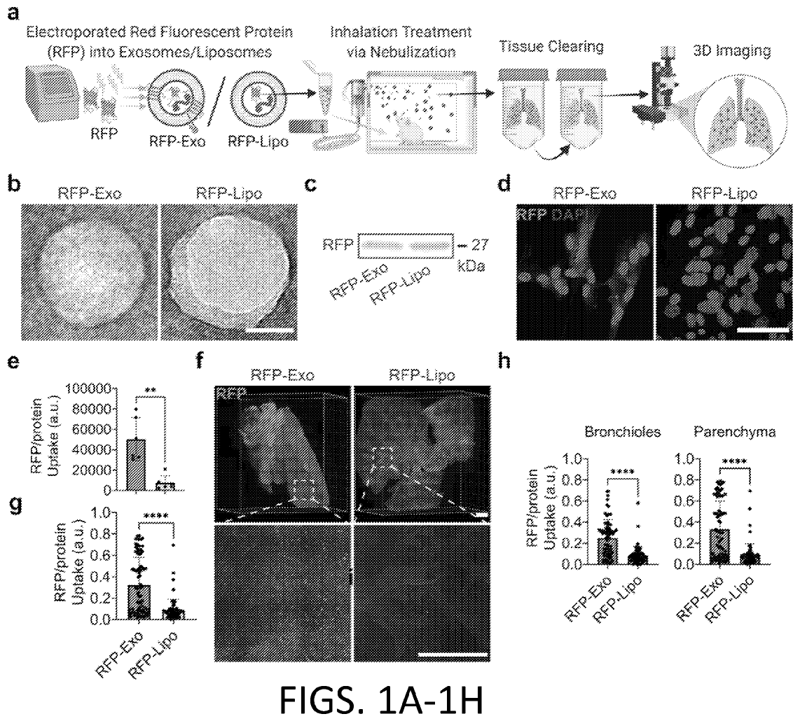

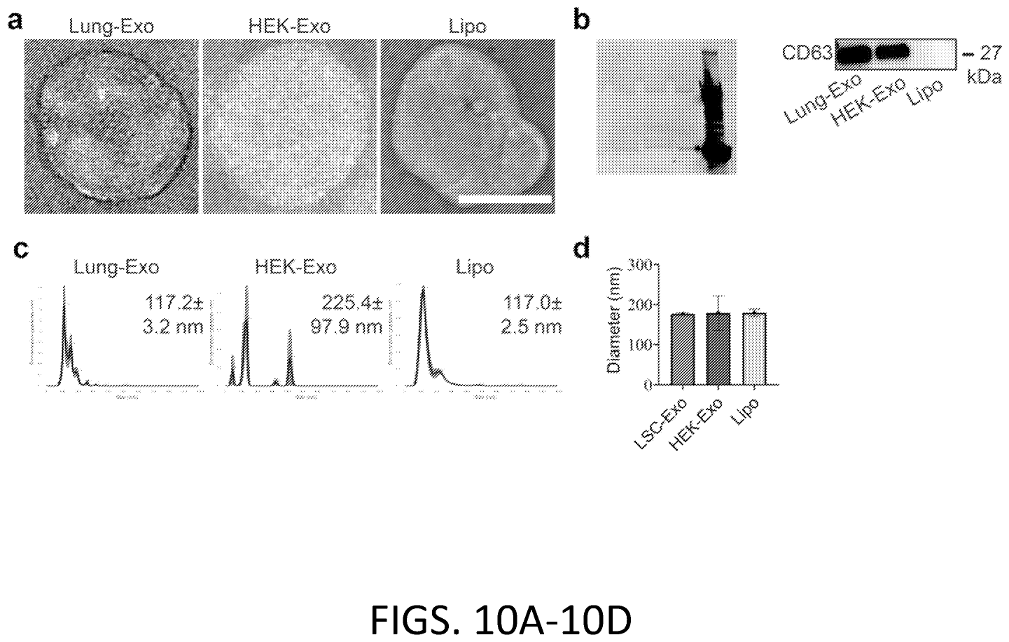

Fabrication and distribution of labeled exosomes and liposomes. Red fluorescent protein (RFP)-labeled lung-derived exosomes (RFP-Exo) and liposomes (RFP-Lipo) were fabricated to generate trackable nanoparticles for biodistribution analysis in the murine lung after inhalation treatment through three-dimensional (3D) imaging (FIG. 1A). The nanoparticles were characterized by transmission electron microscopy (TEM) (FIG. 1B; FIG. 10A), immunoblotting (FIG. 10B), and nanoparticle tracking analysis (NTA) (FIG. 10C). RFP loading was verified by immunoblotting (FIG. 1C). When co-culturing with lung parenchymal cells, RFP-Exo had a 6.7-fold increase in cellular uptake and RFP protein expression than cells cultured with RFP-Lipo (FIGS. 1D and 1E). Next, the biodistribution of nanoparticles in vivo were evaluated through light sheet fluorescence microscopy (LSFM) (FIG. 1F). Healthy mice received a single dose of RFP-Exo or RFP-Lipo via nebulization and were sacrificed after 24 hours. LSFM imaging confirmed nanoparticle delivery to the conducting airways and the deep lung, with an accumulation of RFP-Exo in the upper pulmonary regions. Quantification of nanoparticle delivery to the whole lung demonstrated a 3.7-fold improvement in RFP-Exo retention and uptake than RFP-Lipo (FIG. 1G). Segmentation of the lung into bronchial and parenchymal regions revealed 2.9-fold and 3.8-fold improvements in RFP-Exo retention and uptake than RFP-Lipo, respectively (FIG. 1H). These data confirm that the nanoparticle labeling system maintains nanoparticle integrity, while delivering functional and translatable cargo after jet nebulization. In vitro and in vivo analysis suggest superior retention and cellular uptake of exosomes over liposomes in the lung.

### Example 2

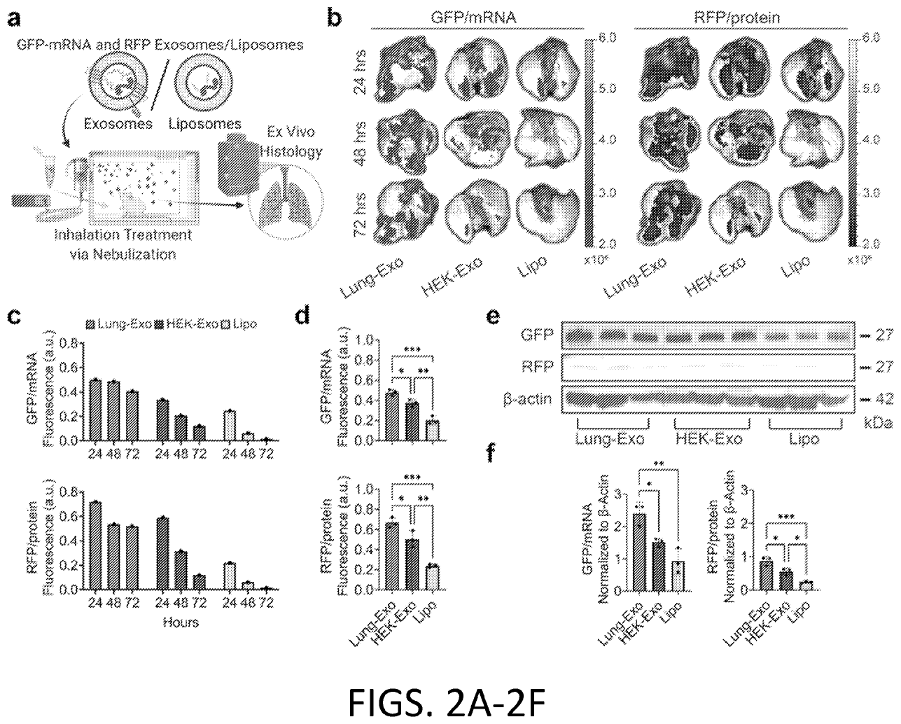

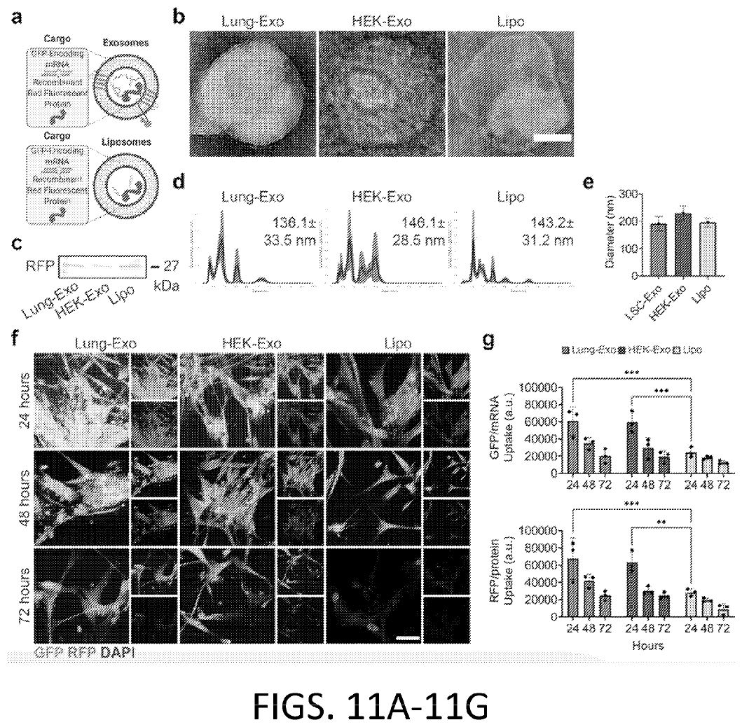

Nebulized lung-derived exosomes have superior distribution and retention in the murine lung. To test if mRNA and protein drugs can be delivered by inhalation, a green fluorescent protein (GFP)-encoding mRNA and RFP protein were loaded into lung-derived exosomes (Lung-Exo), HEK-derived exosomes (HEK-Exo), and liposomes (Lipo) to assess the differences in biodistribution of different cargo after nanoparticle inhalation (FIG. 11A). Nanoparticle characterization and loading were confirmed by TEM (FIG. 11B), immunoblotting (FIG. 11C), and NTA (FIGS. 2E and 2D). Lung-Exo and HEK-Exo had significantly greater mRNA (2.5-fold and 2.4-fold) and protein cargo (2.4-fold and 2.2-fold) uptake by lung parenchymal cells than their liposome counterpart after 24 hours, respectively (FIGS. 2F and 2G). This suggests enhanced cellular targeting by biologically-derived nanoparticles than synthetic nanoparticles.

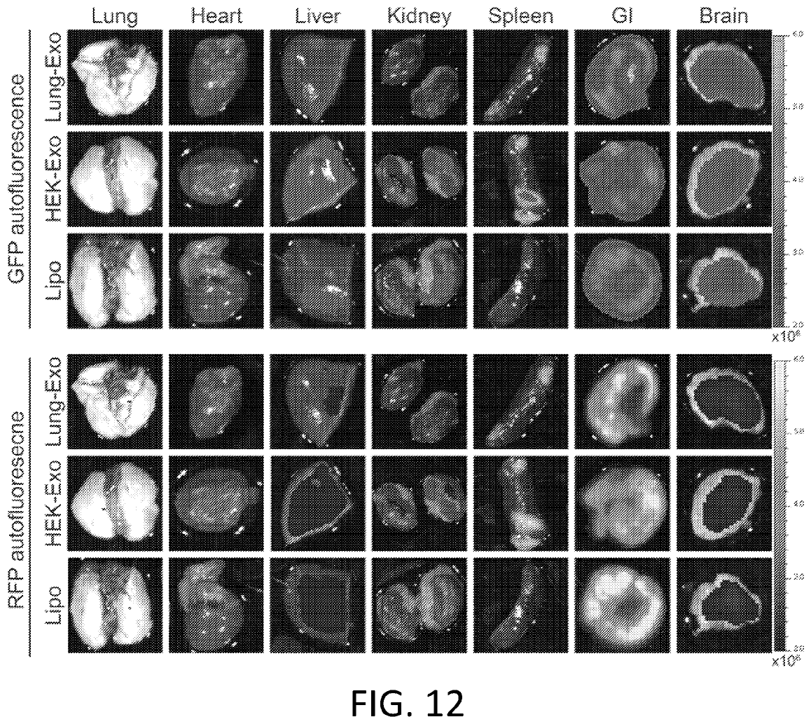

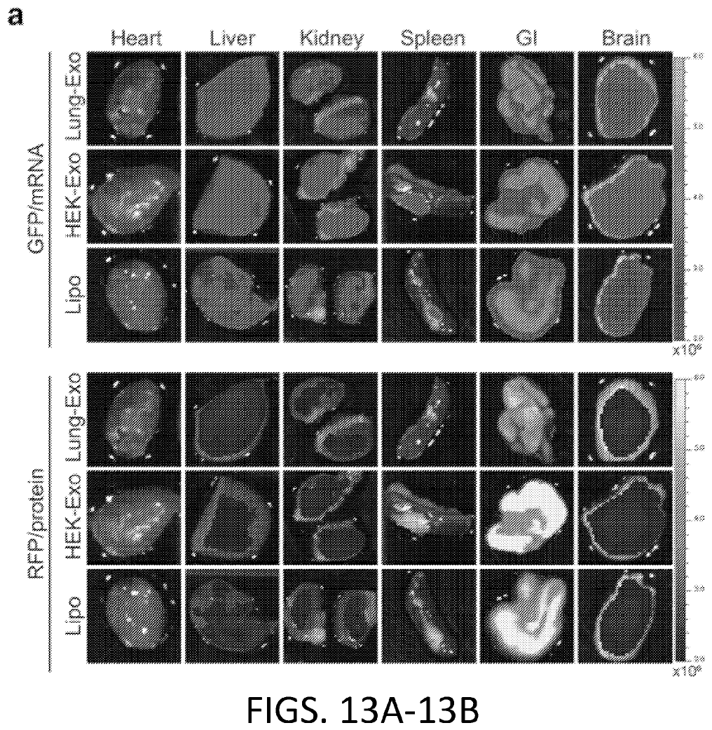

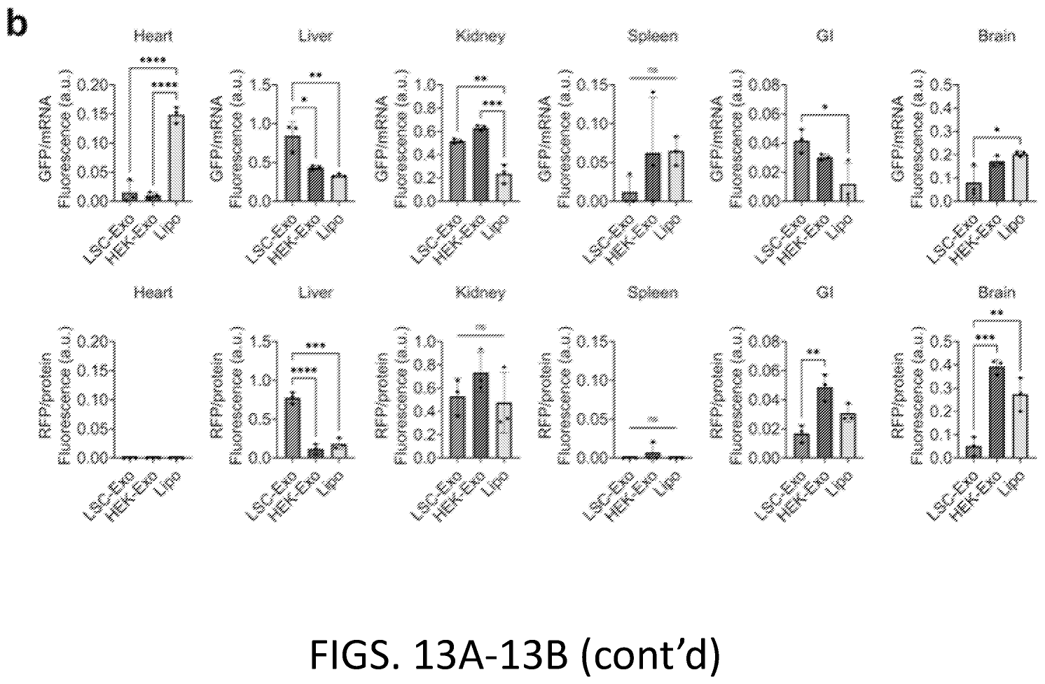

Next, the fluorescently-labeled exosomes and liposomes were tested in vivo by nebulizing a single dose of Lung-Exo, HEK-Exo, and Lipo to healthy mice to evaluate their distribution and retention in the lung (FIG. 2A). Ex vivo imaging revealed the distribution of nanoparticles at 24, 48, and 72 hours (FIG. 2B), with notable clearance or degradation of liposomes over time (FIG. 2C). At 24 hours, Lung-Exo had 1.3-fold and 2.4-fold greater mRNA distribution, and 1.3-fold and 2.8-fold greater protein distribution than its HEK-Exo and Lipo counterparts, respectively, baselined to their native nanoparticle controls (FIG. 12). mRNA and protein cargo delivery were verified by immunoblots (FIG. 2E), where mice who received Lung-Exo had the highest mRNA translation and protein expression in lung tissue (FIG. 2F). Off-targeted uptake of the nanoparticles was tracked in the other major organs (FIG. 13A), where mice who received inhaled Lipo showed rapid clearance and uptake through the pulmonary circulation into the heart (FIG. 13B). Interestingly, Lung-Exo showed significant metabolism to the liver (FIG. 13B). Previous studies have demonstrated significant improvement in liver function through decreased alanine transaminase (ALT) enzymes and decreased serum monocyte chemoattractant protein-1 (MCP-1) in bleomycin rats who received nebulized Lung-Exo, suggesting that Lung-Exo bioavailability may provide an enhanced reduction in systemic inflammation. Together, these data suggest the superior distribution and retention of exosomes in the lung than liposomes. Notably, exosomes of lung origin outperformed the HEK exosome control by having greater nanoparticle distribution, retention, mRNA translation, and protein expression in the lung. These suggest the enhanced bioavailability of Lung-Exo as an inhaled therapeutic and drug delivery vesicle for respiratory diseases.

### Example 3

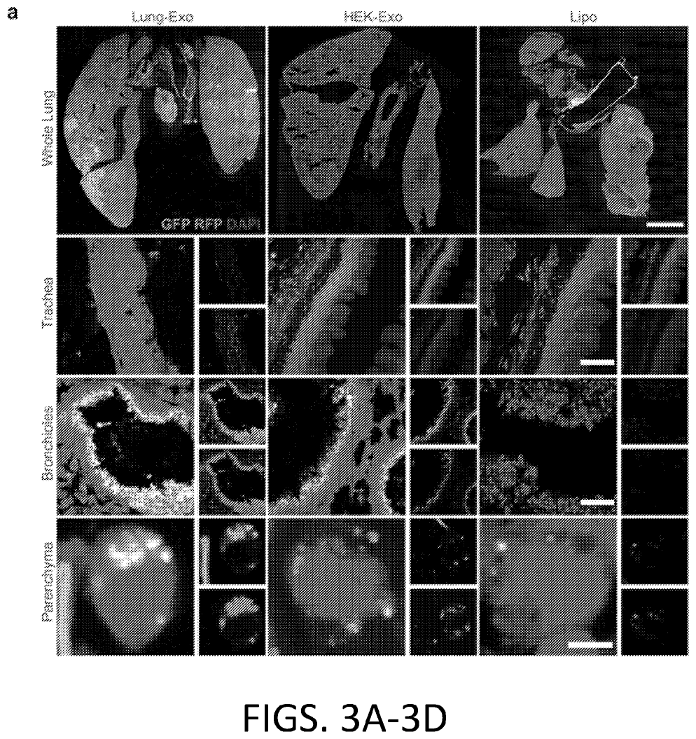

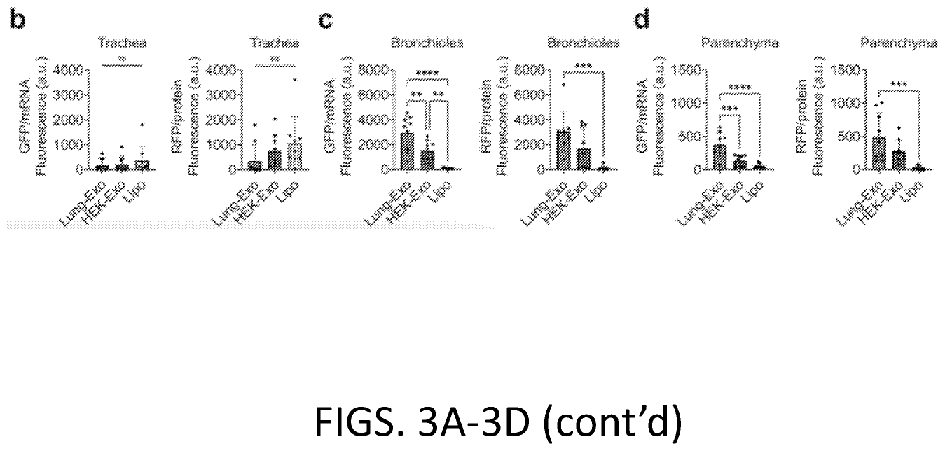

Lung-derived exosomes have superior delivery of mRNA and protein to the bronchioles and parenchyma. Nanoparticles are an attractive inhaled therapeutic in that their innate size distributions (<5 μm) are immediately respirable and allow for alveolar deposition. To track inhaled nanoparticles into the deep lung, the whole lung was segmented into its three main areas: the trachea, bronchioles, and parenchyma (FIG. 3A). Liposomes showed trends of tracheal deposition (FIG. 3B), while exosomes showed significantly greater deposition into the bronchioles (FIG. 3C) and parenchyma (FIG. 3D). Lung-Exo had the greatest exosomal protein expression in the bronchioles (24.1-fold) and parenchyma (22.9-fold) compared to Lipo. Notably, Lung-Exo had the greatest exosomal mRNA translation in the bronchioles (1.9-fold and 27.5-fold) and parenchyma (2.8-fold and 7.2-fold) than both HEK-Exo and Lipo, respectively. These data suggest that exosomal mRNA delivery and clinical translation may be significantly impacted by its nanoparticle phenotype. The native lung signature of Lung-Exo may provide superior delivery and retention of cargo components to the lung than exosomes derived from different derivations or synthetic nanoparticles.

### Example 4

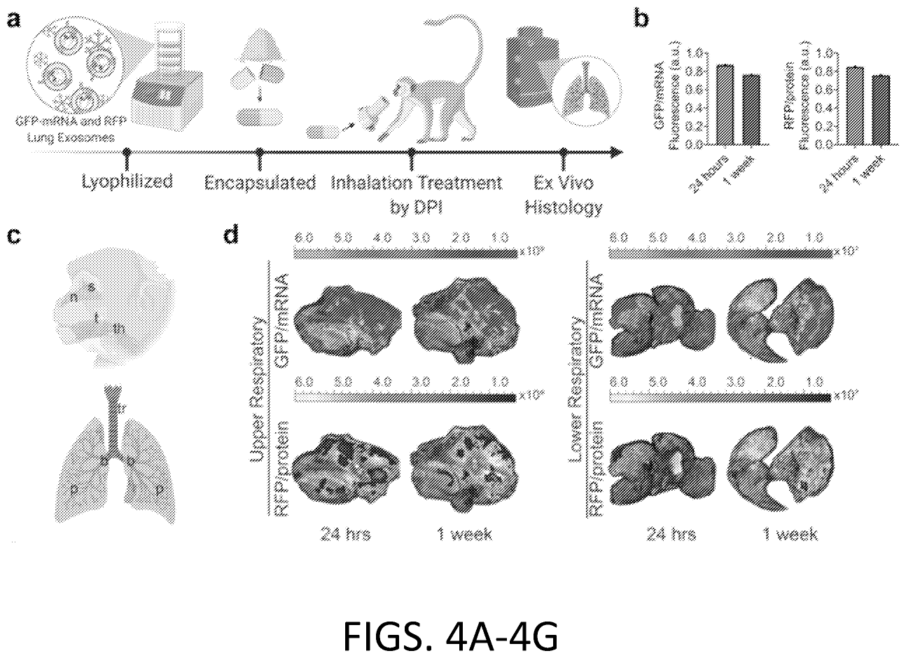

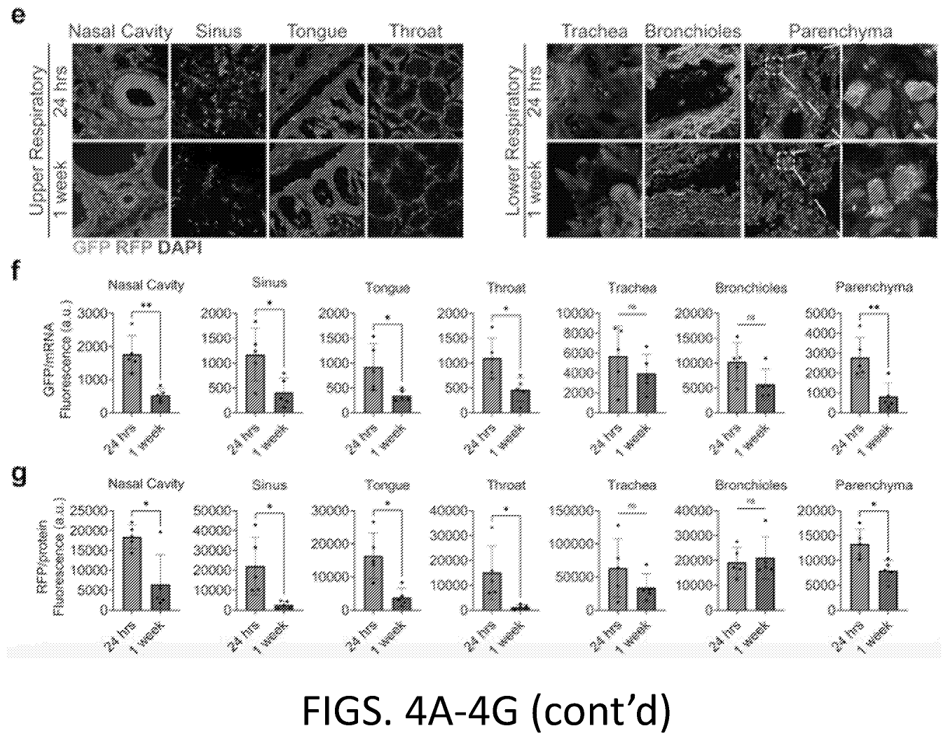

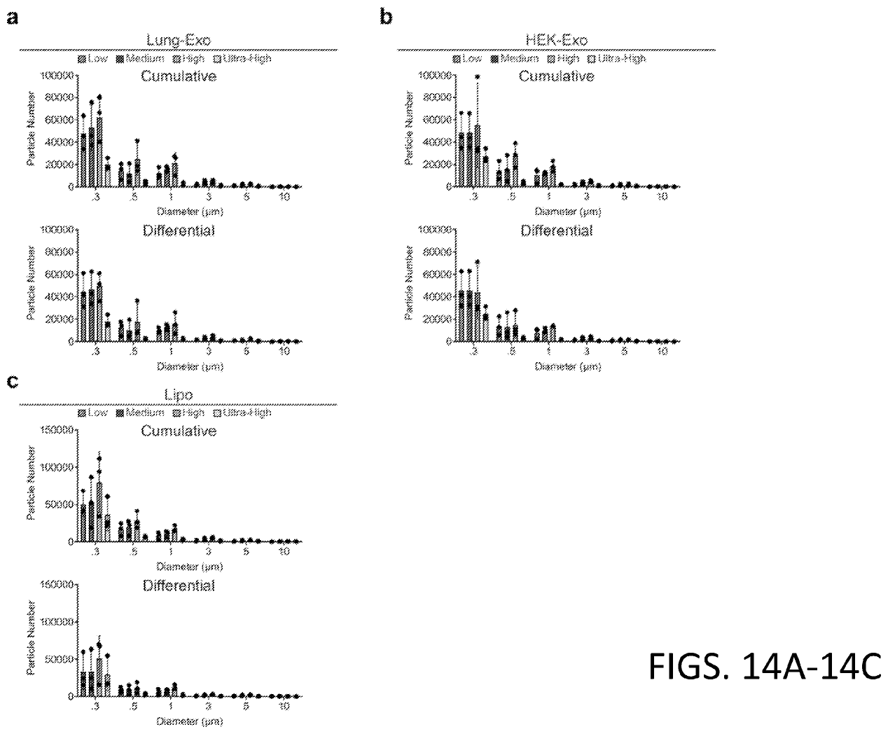

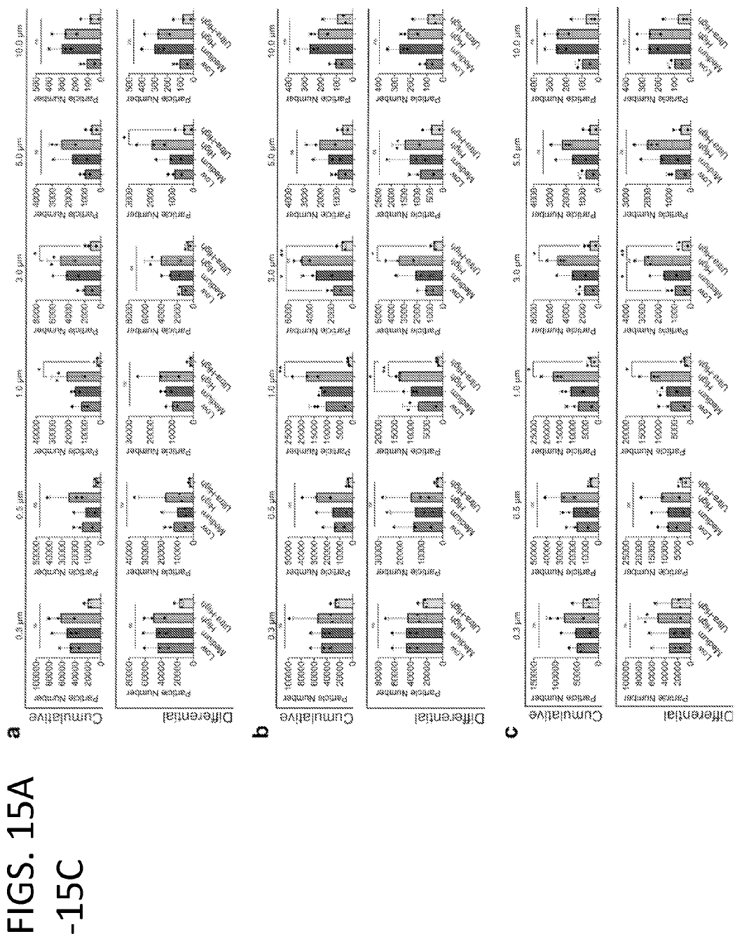

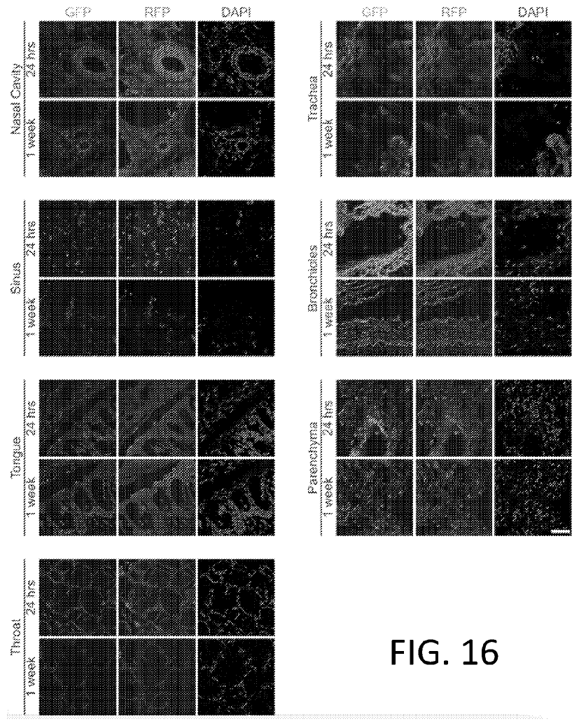

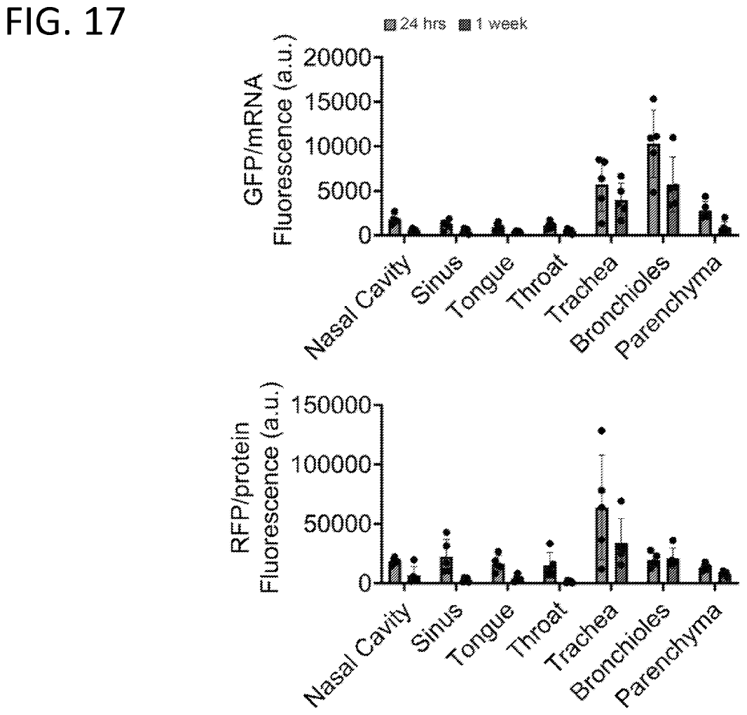

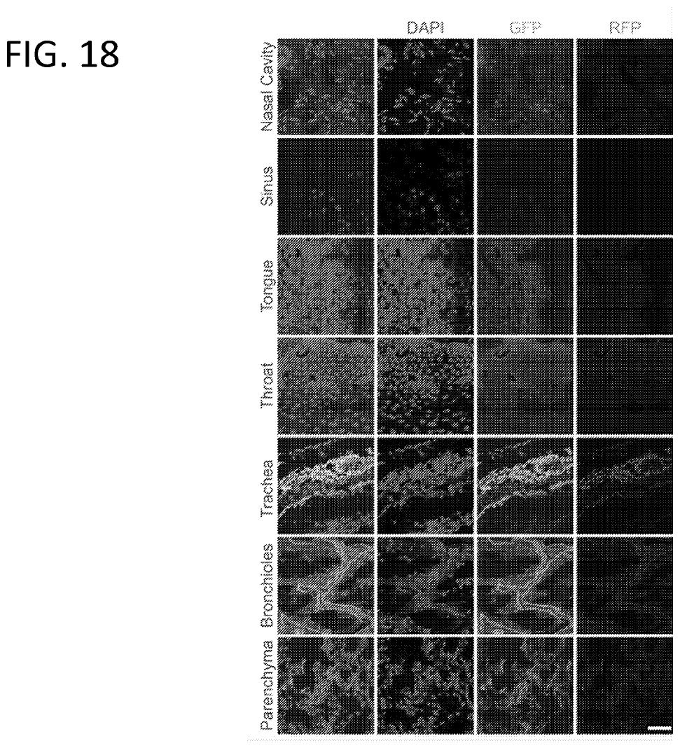

Distribution of lung-derived exosomes via dry powder inhalation in African green monkeys. Dry powder inhalers (DPI) offer an at-home or on-the-go electronic-free administration of therapeutics through a user-friendly device designed specifically for pulmonary disease treatment, providing local drug delivery and reducing side effects associated with IM and oral drugs. Therefore, the liquid nanoparticle suspensions were reformulated into dry, lyophilized powder for dry powder inhalation administration. Lung-Exo, HEK-Exo, and Lipo were lyophilized and encapsulated into commercially-available hydroxypropyl methylcellulose (HPMC) capsules. Exosome biodistribution was tracked in the AGM by delivering a single dose of lyophilized Lung-Exo and sacrificing after 24 hours and 1 week for further analysis (FIG. 4A). A commercially available RS01 high-resistance DPI was selected for its overall greater output of aerosols within the respirable fraction (FIG. 14) and consistent particle distributions across varying nanoparticles (FIGS. 15A-15C). Ex vivo imaging revealed a similar biodistribution of exosomal mRNA and protein cargo throughout the lung (FIGS. 4B and 4D). Lung-Exo were maintained in the lung 1-week after administration (FIG. 4B). Further immunostaining analysis of the upper and lower respiratory tracts confirmed exosome delivery (FIGS. 4C and 4E; FIG. 16) and the greatest nanoparticle deposition into the lung (FIG. 17). Exosomal mRNA (FIG. 4F) and protein (FIG. 4G) were significantly cleared from the upper respiratory and parenchymal regions. Tracheal and bronchial regions maintained similar GFP and RFP fluorescence, which can be explained by the autofluorescent nature of airway tissue (FIG. 18A). Overall, Lung-Exo are distributable in the simian upper and lower respiratory tracts through dry powder inhalation.

### Example 5

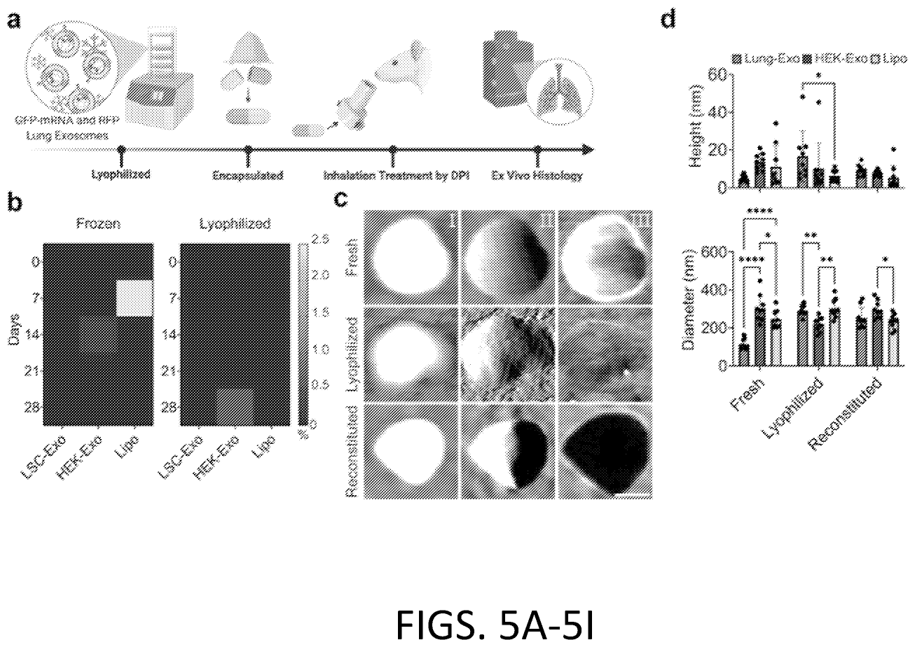

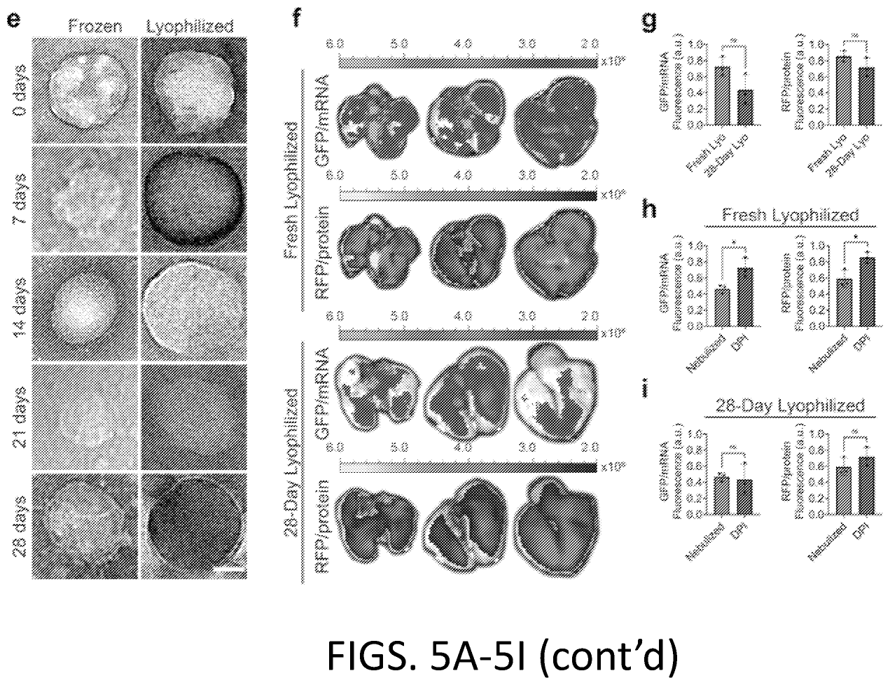

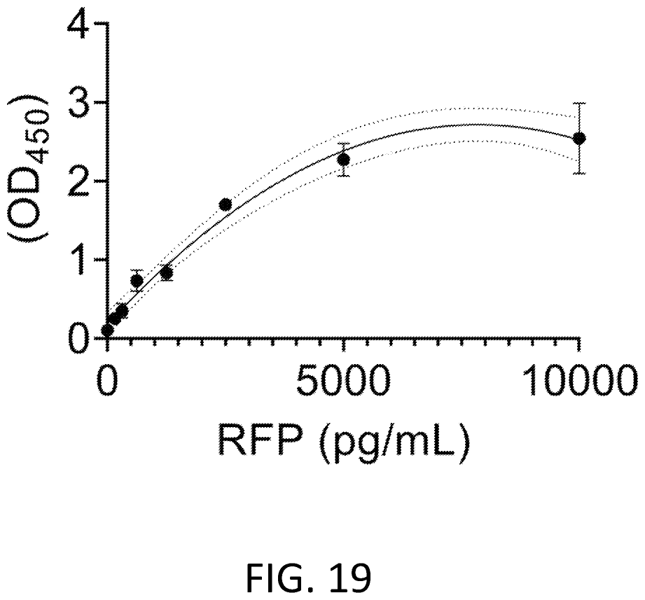

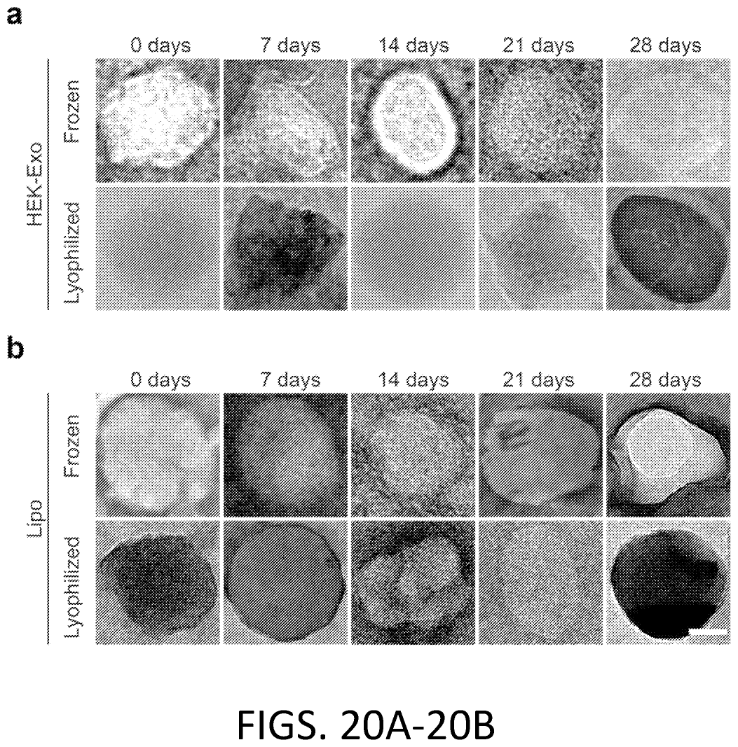

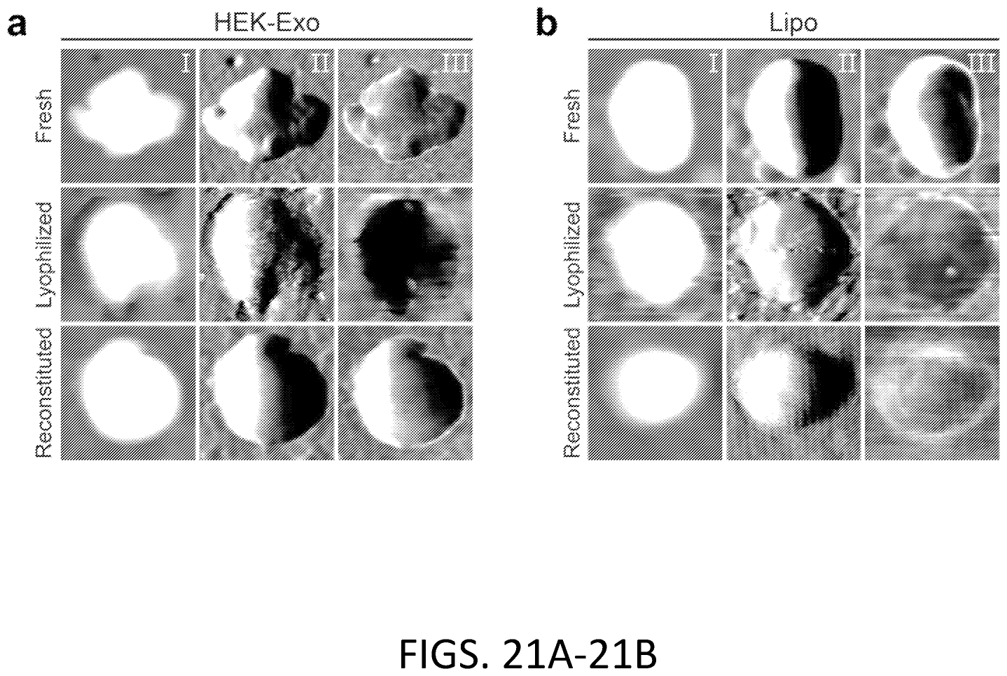

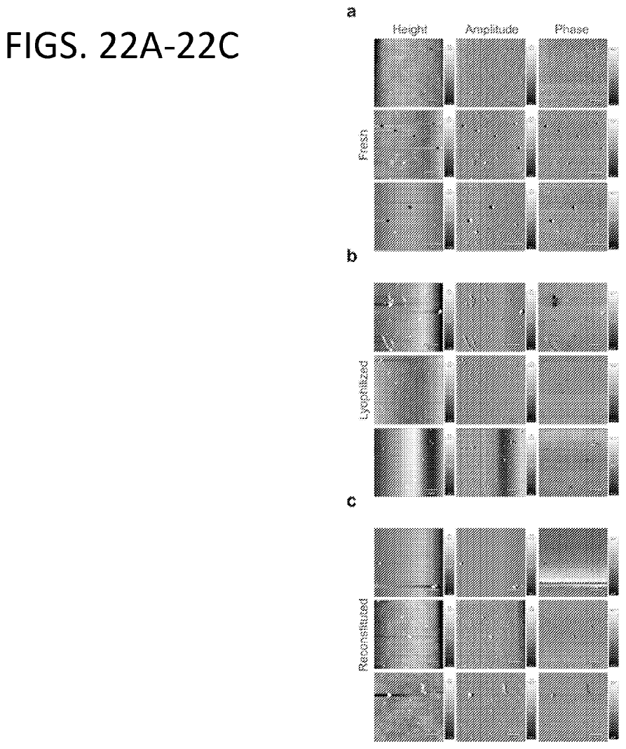

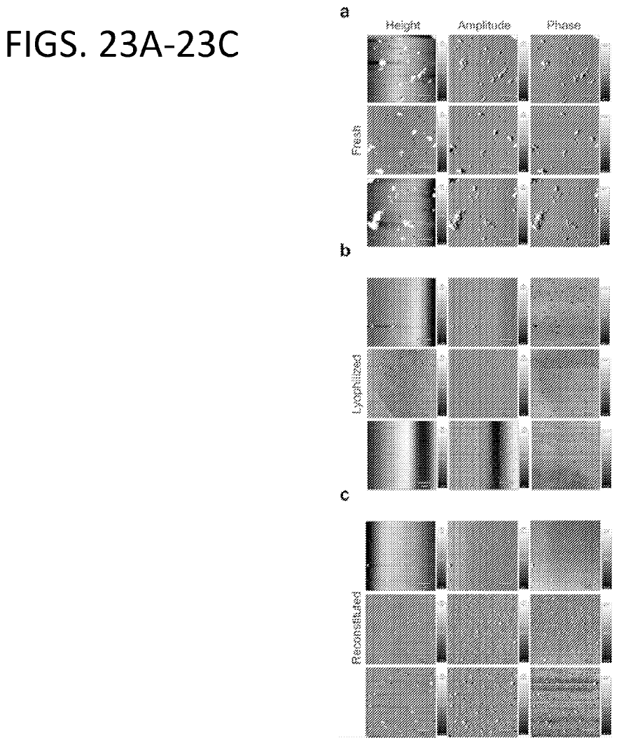

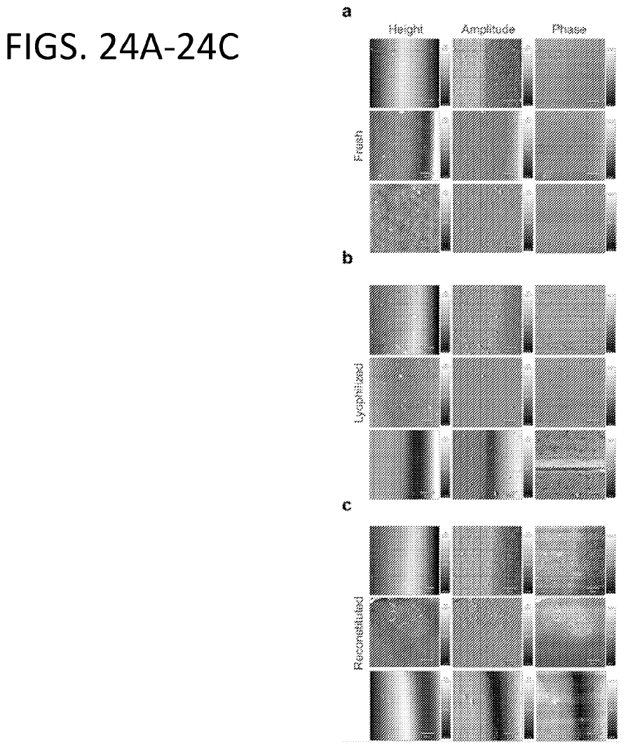

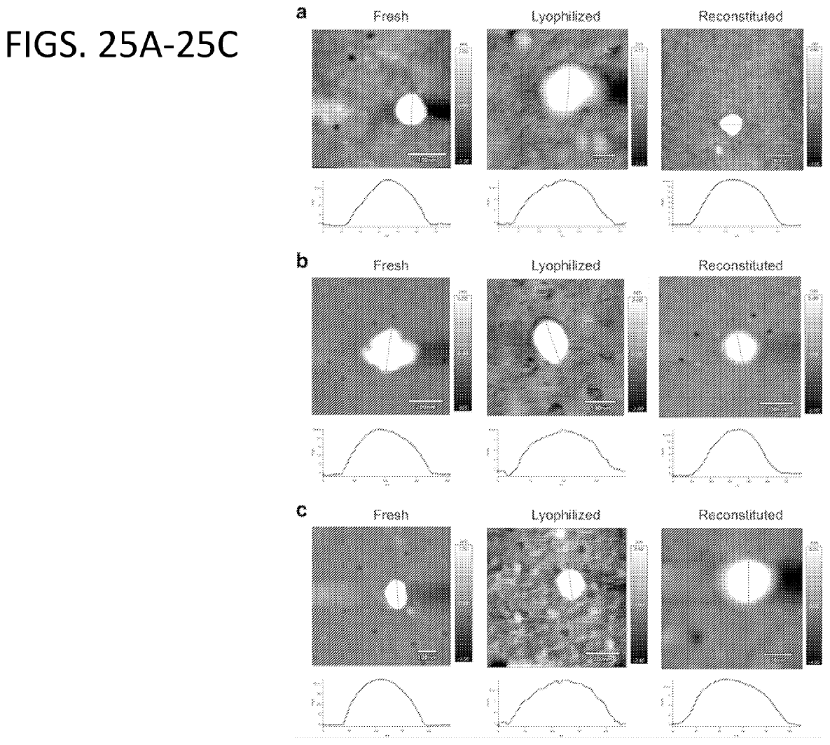

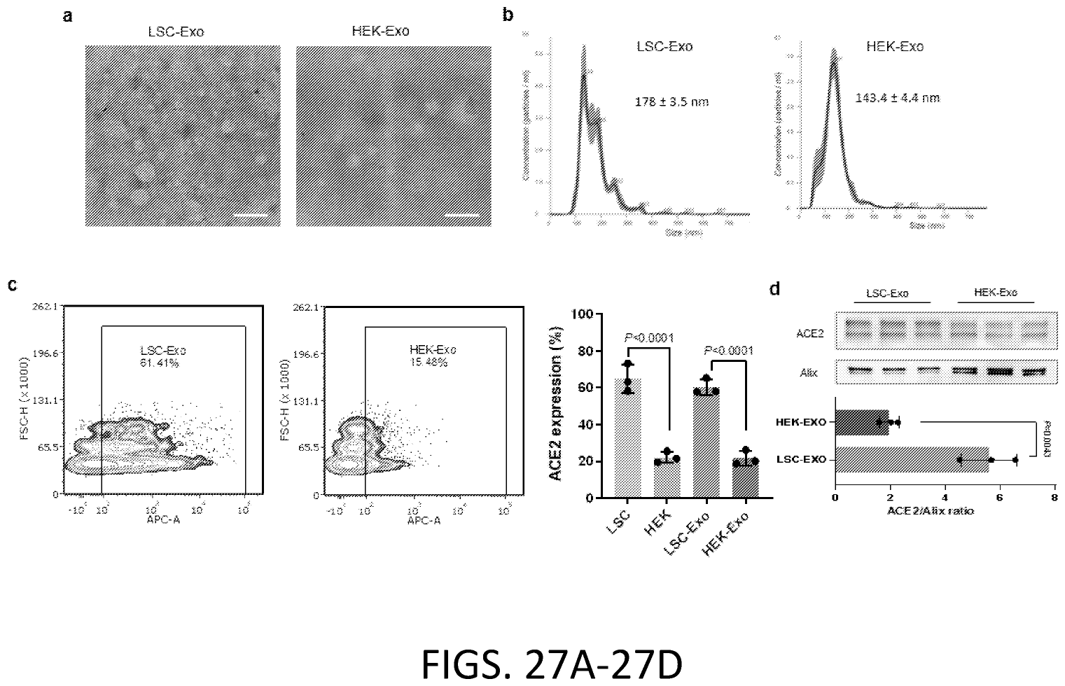

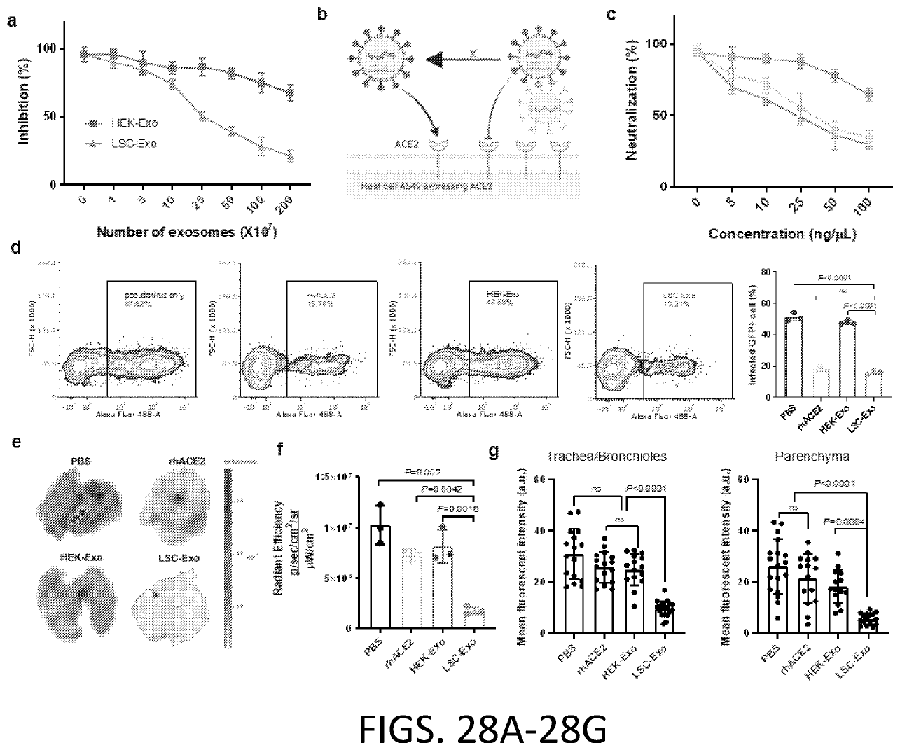

Lung-derived exosomes are room-temperature stable and distributable in dry powder formulation in the murine lung. Room-temperature formulation of therapeutics circumvents major limitations in traditional IM vaccine delivery: deep-freezing storage, expensive shipment, and healthcare professional administration. The efficacy and stability of room-temperature lyophilized Lung-Exo were verified up to 28 days in the murine lung (FIG. 5A). To verify dry powder nanoparticle stability and shelf life, lyophilized nanoparticle cargo leakage was tested by an enzyme-linked immunosorbent assay (ELISA), where nanoparticles had less than 2.4% of total pg/mL cargo leakage at day 28 of room-temperature storage (FIG. 5B; FIG. 19). Next, the morphology of nanoparticles was evaluated across their fresh and lyophilized formulations, as well as lyophilized powder reconstituted in water (reconstituted), to mimic rehydration of dry powder by saliva and mucus. TEM (FIG. 5E; FIG. 20) and atomic force microscopy (AFM) verified that reformulation and rehydration did not affect nanoparticle membrane integrity (FIG. 5C; FIGS. 21A and 21B), but affected size distributions through clumping (FIGS. 22-24). Lyophilization increased nanoparticle height and diameter (FIG. 5D), but remained as small respiratory droplets upon reconstitution. Cross-section measurement curves demonstrate a restoration of membrane “smoothness” in reconstituted nanoparticles, mimicking fresh formulation (FIG. 25). The lyophilized Lung-Exo were delivered via DPI, where ex vivo images (FIG. 5F) of mouse lungs who received fresh (Fresh Lyophilized) and 28-day-old (28-Day Lyophilized) dry powder Lung-Exo had no significant difference in exosomal mRNA and protein distribution (FIG. 5G). Compared to nebulized exosome biodistribution, both fresh (FIG. 5H) and 28-day-old (FIG. 5I) lyophilized exosomes have trends of greater pulmonary distribution 24-hours after administration. Lyophilized Lung-Exo is a room-temperature stable exosome formulation that can deliver functional and translatable cargo via dry powder inhalation.

### Example 6

Lung-derived exosomes efficiently penetrate mucus. Delivery of inhaled therapeutics must penetrate the lung's protective mucus lining to provide pulmonary bioavailability. Lung-derived exosomes (Lung-Exo) were compared against HEK-derived exosomes (HEK-Exo) and liposomes (Lipo), to determine if nanoparticle derivation affected mucus penetrance. To test this, a model of the human airway was used at the air-liquid interface (FIG. 26A), with human mucus-secreting bronchial epithelial cells lining the transwell membrane and human lung parenchymal cells lining the well (FIG. 26B). Immunostaining confirmed the mucus lining in the transwell membrane and delivery of DiD-labeled nanoparticles (FIG. 26C). Quantification of nanoparticle penetrance into the wells revealed the greatest uptake of Lung-Exo (FIG. 26D), with the highest percentage of cellular uptake by lung parenchymal cells (FIG. 26E) by 24 hours. Likewise, Lung-Exo had the least entrapment by the mucus-lined membrane (FIG. 26F) and the lowest percentage of cellular uptake by bronchial epithelial cells (FIG. 26G). These data confirm mucus penetrance of the nanoparticles and suggest that Lung-Exo can most efficiently evade mucoadhesion, overcoming the lung's natural defense mechanism and allowing for parenchymal bioavailability.

### Example 7

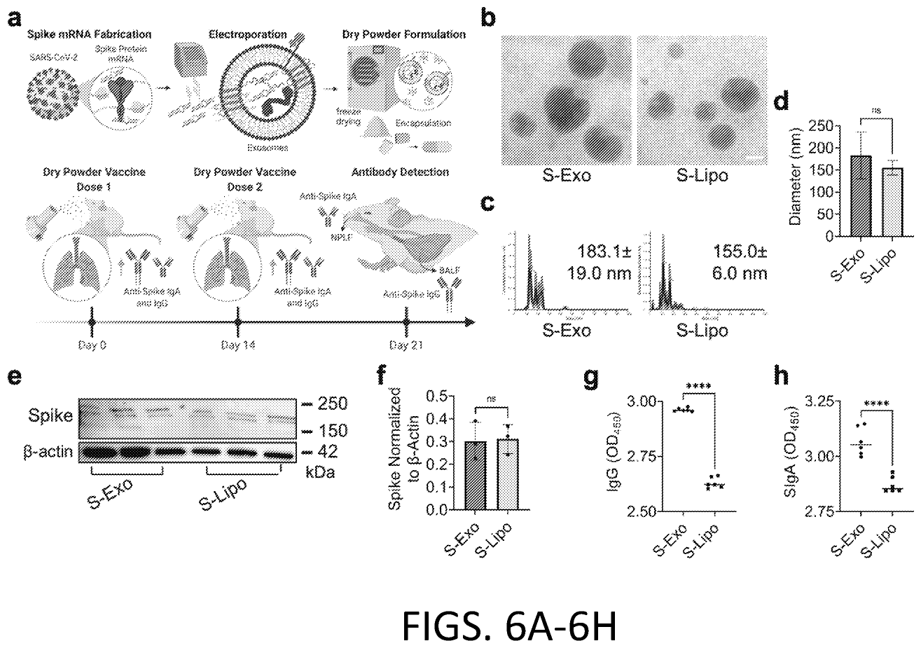

SARS-CoV-2 spike loaded exosomes elicit antibody protection through dry powder inhalation. In this study, the therapeutic potential of lyophilized Lung-Exo was tested as an inhaled vaccine against COVID-19. mRNA encoding the severe acute respiratory coronavirus 2 (SARS-CoV-2) spike(S) protein was loaded into Lung-Exo and Lipo and formulated for dry powder inhalation, generating S-Exo and S-Lipo, respectively (FIG. 6A). To confirm long-term storage efficiency, S-Exo and S-Lipo were stored for one month at room temperature prior to characterization and mouse vaccination. Nanoparticle vaccine integrity was verified by TEM (FIG. 6B) and NTA (FIGS. 6C and 6D). S protein-encoding mRNA loading and in vitro cellular translation were verified by immunoblotting (FIG. 6E), where S-Exo and S-Lipo had similar results (FIG. 6F).

The inhaled vaccine was then tested in healthy mice who received two doses of S-Exo or S-Lipo via DPI. The mice were sacrificed one week after the second dose and bronchoalveolar lavage fluid (BALF) and nasopharyngeal lavage fluid (NPLF) were collected to assess anti-spike IgG and SIgA antibody production, respectively (FIG. 6A). ELISAs revealed that both S-Exo and S-Lipo produced sufficient IgG antibodies (>2.18325; FIG>6G) and SIgA antibodies (>1.14595; FIG. 6H) to induce neutralizing antibody responses. However, dry powder inhalation of S-Exo produced significantly higher amounts of antibodies than S-Lipo (FIGS. 6G and 6H). This suggests that S-Exo would have superior protection against SARS-CoV-2 infection over S-Lipo as an inhaled vaccine. Coupled with enhanced mucus penetrance, S-Exo may further facilitate passive IgG defusal into the pulmonary epithelial lining fluid. The features of Lung-Exo have significant advantages over its synthetic nanoparticle counterpart, suggesting that exosomes contain additional therapeutic benefits regarding pulmonary targeting, retention, and immune responses. Biological nanoparticles such as exosomes can be exploited as inhaled drug delivery vehicles, to maximize drug targeting, delivery, and therapeutic efficacy.

### Example 8

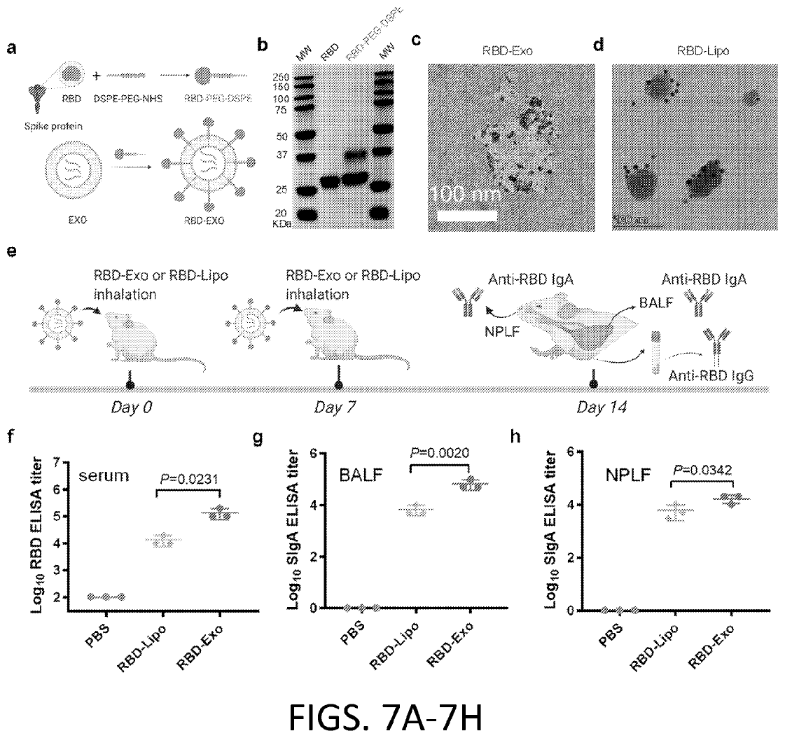

Receptor binding domain (RBD) of Spike protein conjugated with exosomes (RBD-Exo) elicited superior antibodies production over RBD conjugated with liposome (RBD-Lipo). A virus-like particle vaccine was developed by conjugating receptor binding domain of spike protein on the surface of LSC-Exo (RBD-Exo). That emulates the morphology of the native virus. To synthesize RBD-Exo VLP, RBD antigens were firstly conjugated with [1,2-Distearoyl-sn-glycero-3-phosphoethanolamine-poly (ethylene-glycol)-N-hydroxysuccinimide] (DSPE-PEG-NHS) to form RBD-PEG-DSPE, as demonstrated by SDS-PAGE (FIGS. 7A and 7B). Next, RBD-PEG-DSPE was conjugated on the surface of LSC-Exo and the binding capacity was calculated to 0.52 μg RBD per 10¹⁰ exosomes. Furthermore, RBD conjugated with liposome (RBD-Lipo) was synthesized as a control and determined to be 0.41 μg RBD per 10¹⁰ liposomes. Transmission electron microscopy (TEM) was utilized to characterize RBD-Exo or RBD-Lipo. As illustrated in FIGS. 7C and 7D, gold nanoparticles were conjugation to anti-RBD antibodies to confirm the presence of RBD on the exosome surface and liposome surface, respectively.

The immunogenicity of RBD-Exo or RBD-Lipo was tested in healthy mice who received two doses of immunizations via nebulization (FIG. 7E). The ELISA results revealed that inhalation of RBD-Exo VLPs induced the most RBD-specific IgG antibodies against RBD (FIG. 7F) in the mouse sera, significantly higher than RBD-Lipo nebulization. Furthermore, RBD-Lipo induced a low level of RBD-specific IgA antibodies from both BALF (FIG. 7G) and BALF (FIG. 7H) compared to RBD-Exo immunizations. These results indicated that LSC-exosomes could be exploited as inhaled vaccine vehicles, to enhance the immunogenicity of antigen in relative to liposomes.

### Example 9

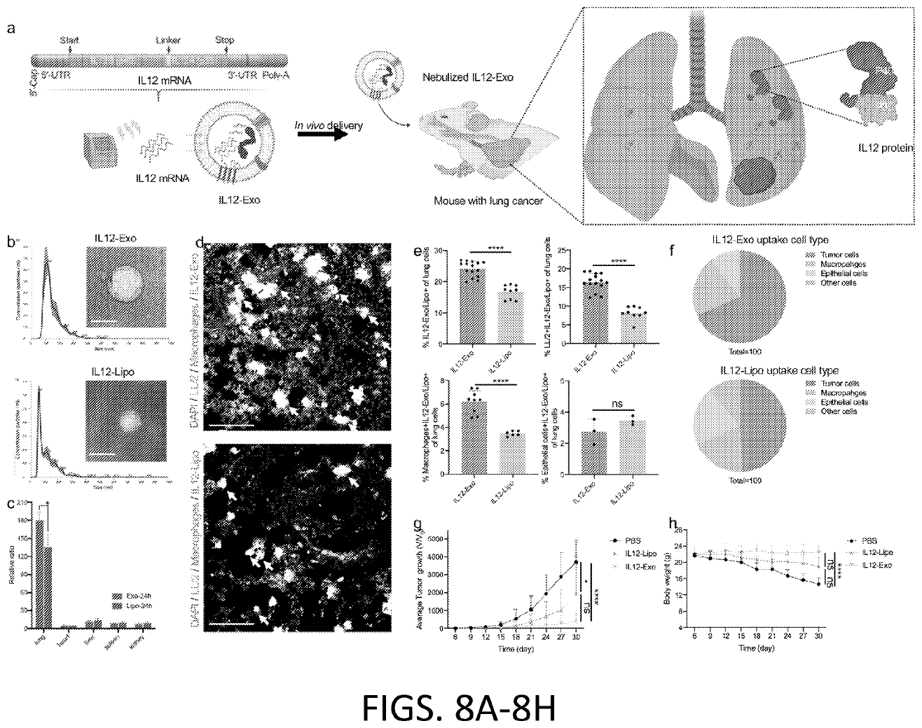

IL12 mRNA loaded HEK-Exo elicit tumor immunotherapy through local inhalation delivery. Based on the superior exosomes accumulation in lungs over liposomes, IL12 mRNA, an immunotherapy regent, loaded HEK-Exo (IL12-Exo) was tested via electroporation for lung cancer treatment (FIG. 8A). IL12 mRNA loaded liposomes (IL12-Lipo) were used as a control. The size for IL12-Exo and IL 12-Lipo were 151.0 nm and 116.2 nm, respectively (FIG. 8B). Their morphologies were also characterized by TEM (FIG. 8B). To specify the distribution of HEK-Exo in organs and in tumor microenvironment (TME), the fluorescence of HEK-Exo or liposome was tested 24 h after the inhalation of DiD labeled IL 12-Exo or IL12-Lipo. Ex vivo imaging results revealed high lung accumulation of IL12-Exo compared to IL 12-Lipo (FIG. 8C). It was also validated by flow cytometry quantified analysis, the uptake efficiency of IL 12-Exo was 24.2% compared to 16.7% of IL12-Lipo in total lung cells (FIG. 8D). In TME, more than 68% IL12-Exo were internalized by tumor cells, while the rest were mainly by macrophages and epithelial cells. In comparison, IL12-Lip had low tumor targeting (<50% uptake by tumor cells) (FIGS. 8E and 8F). Experiments were also conducted to test the anti-tumor effects of IL12-Exo in vivo. Mouse with lung tumor model was established at day 0, then mice were treated with IL12-Exo, IL 12-Lipo or PBS, respectively, at day 6, 9 and 12. IL12-Exo could dramatically inhibited tumor growth among 30 days, while IL12-Lipo showed mild inhibition ability (FIG. 8G). In addition, IL12-Exo did not induce body loss (FIG. 8H). Those results demonstrated the superior tumor homing and immunotherapy capability of IL12-Exo.

### Example 10

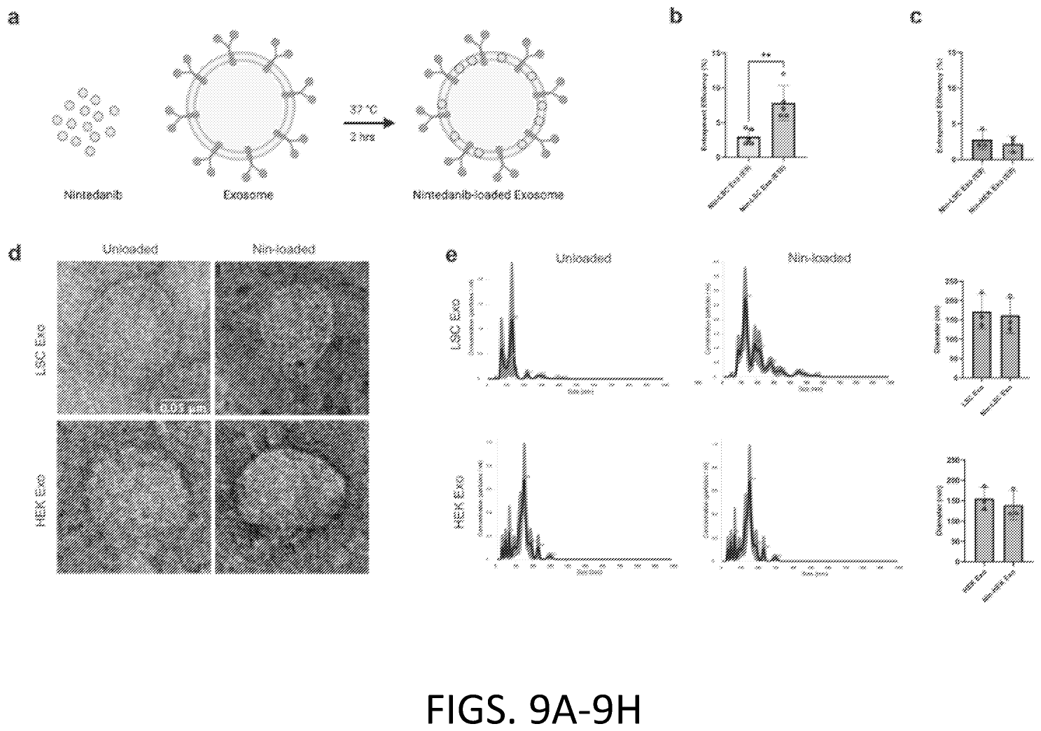

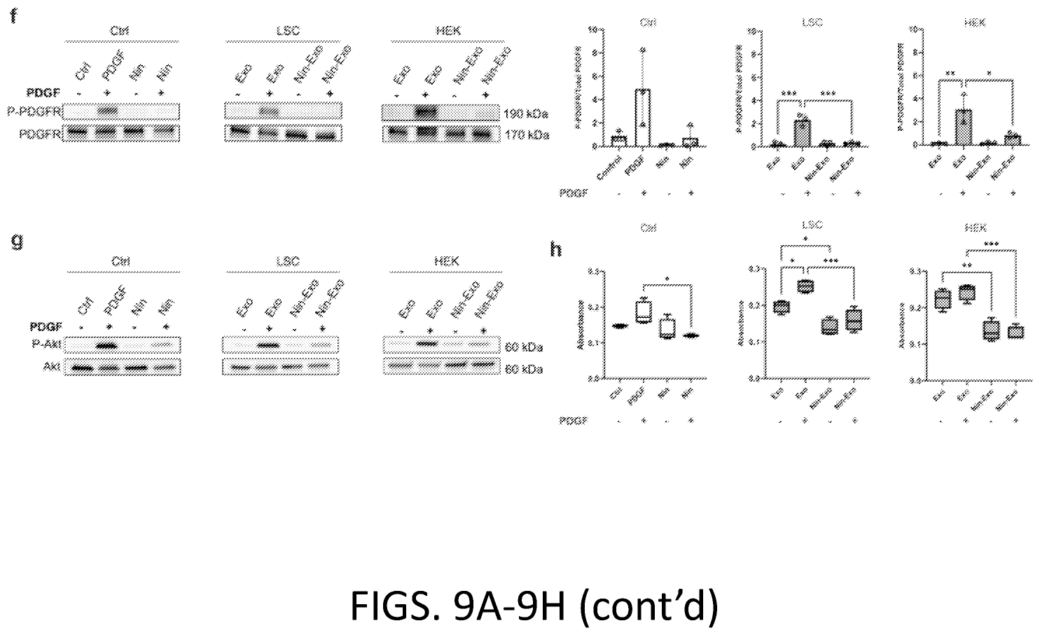

Nintedanib-loaded LSC exosomes act as drug carriers to inhibit idiopathic pulmonary fibrosis lung fibroblast activation and proliferation. Exosomes were utilized as vehicles to deliver Nintedanib, an FDA-approved antifibrotic small molecule drug. Nintedanib was passively loaded into LSC (Nin-LSC Exo) via incubation and compared against exosomes from the widely used HEK-293 cell line (Nin-HEK Exo) (FIG. 9A). As measured by HPLC, drug encapsulation efficiency improved significantly with a 10-fold increase in exosome ratio from average of 3.36% to 6.5% in LSC exosomes (FIG. 9B). LSC and HEK exosomes presented comparable encapsulation efficiency at 1 billion particles (FIG. 9C). Sphere-shaped morphology of exosomes was maintained (FIG. 9D) and exosome diameter did not significantly change following drug loading (FIG. 9E). Nintedanib-loaded exosomes effectively inhibited the phosphorylation of PDGF Receptor α/β in the PDGF-induced primary idiopathic pulmonary fibrosis (IPF) lung fibroblasts which was comparable to the Nintedanib treatment alone (FIG. 9F). Similar to Nintedanib function, Nintedanib-loaded exosomes downregulated phosphorylated-Akt, a downstream signaling molecule of PDGF pathway (FIG. 9G). Furthermore, treating PDGF-induced IPF lung fibroblasts with Nintedanib-loaded exosomes significantly suppressed cell proliferation in vitro (FIG. 9H). The present data suggest that exosomes derived from lung cells can perform as functional drug delivery vehicles similar to exosomes derived from HEK cells. In vivo efficacy of inhaled Nintedanib-loaded LSC exosomes in IPF mouse model will be examined as the next phase of the study.

### Example 11

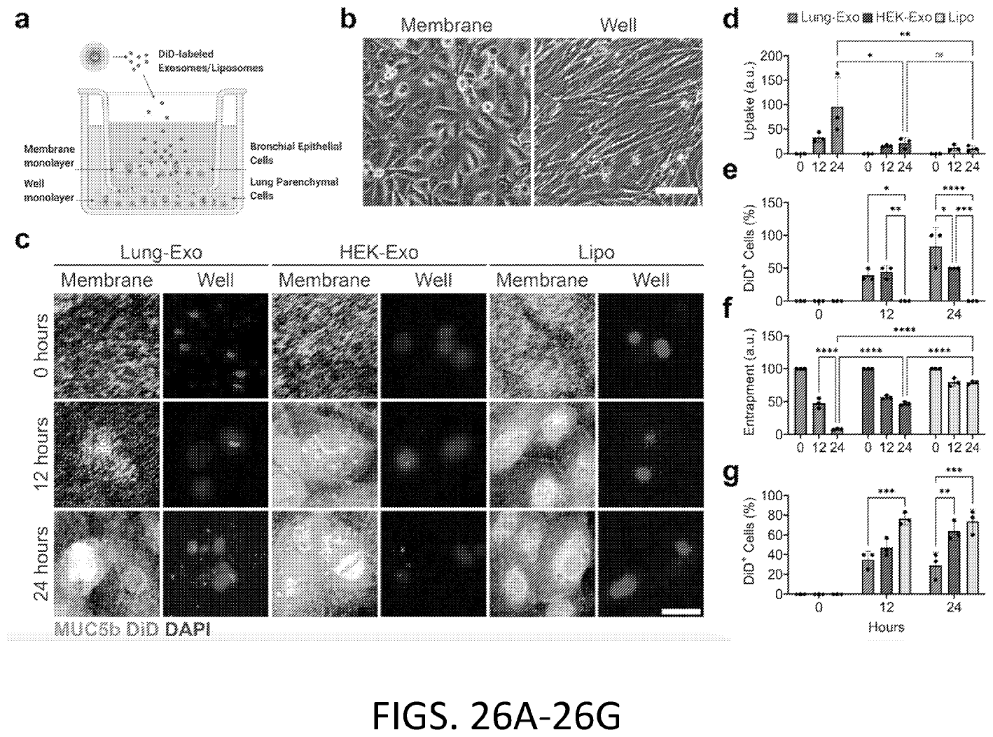

Recent studies hint at the potential of cellular membrane-derived nanovesicles (NVs) displaying hACE2 that compete with host cells for SARS-CoV-2 binding, protecting the host cells against SARS-CoV-2 infection. Previous work was done to develop hACE2 NVs derived from healthy human lung spheroid cells (LSC) that could serve as decoys to neutralize SARS-CoV-2 and trigger subsequent phagocytosis by macrophages to clear the virus in a non-human primate model. Additionally, engineered extracellular vesicles with enriched hACE2 expression have been demonstrated to protect mice against SARS-CoV-2 lung inflammation efficiently. Although promising, their further clinical translations were hindered by the random directions of hACE2 on cell membrane-derived NVs and the potential risks of gene engineering. Interestingly, exosomes and viruses employ similar endosomal sorting pathways and mechanisms, endowing exosomes with the potency to be a new therapeutic reagent for targeting, binding, and suppressing cellular uptake of various viruses including SARS-CoV-2. Furthermore, by sharing surface receptor proteins, microRNA, and DNA with their parental cells, lung-derived exosomes would harness superior homing-target ability towards lung over their exogenous counterparts. Further, LSC as a cell therapy was developed from initial rodent studies to an ongoing phase 1 clinical trial (NCT04252167). LSC represent nature mixtures of resident lung epithelial cells consisting of both types I and II pneumocytes and mesenchymal cells. Being resident lung cells, they express ACE2 endogenously; therefore, it is speculated that LSC-Exo could carry the parental cell's ACE2, target lung, and confer protection against SARS-CoV-2 infection (FIG. 27A).

Experiments were conducted to systematically assess the efficacy of LSC-Exo for prophylaxis of SARS-CoV-2 infection and SARS-CoV-2 VOC. Since SARS-CoV-2 infection basically starts in the nasal cavity, followed by aspiration of the viral inoculum from the oropharynx into the lower respiratory tract, inhalation delivery of LSC-Exo was performed to endue effective protective benefit on the affected sites. Direct evidence is provided that LSC-Exo express enriched hACE2 and could cross the air-blood-barrier to reach and accumulate in trachea, bronchioles, and deep lung parenchyma after nebulization. Importantly, LSC-Exo prevented SARS-CoV-2 infection in Syrian hamsters, a model of severe COVID-19 disease, by a drastically reduced viral load, diminished lung inflammation, and dampened viral pneumonia. More importantly, it was demonstrated that LSC-Exo preserve the neutralizing capacity against UK and Delta pseudoviruses.

Characterization of LSC-Exo. Both LSC-derived exosomes (LSC-Exo) and HEK cell-derived exosomes (HEK-Exo) were collected purified. Both of which exhibited a similar morphology and size as measured by transmission electron microscopy (TEM; FIG. 27A) and nanoparticle tracking analysis (NTA; FIG. 27B). Experiments were conducted to evaluate the level of ACE2 on LSC-Exo and HEK-Exo by flow cytometry and immunoblotting assay. As illustrated in FIG. 27C, LSC-Exo exhibited significantly higher ACE2 expression than HEK-Exo, in line with the results of their parent cells. Immunoblotting assay further validated that LSC-Exo, but not HEK-Exo, expressed enriched hACE2 (FIG. 27D).

LSC-Exo neutralize SARS-CoV-2 pseudovirus in vitro and in vivo. To evaluate the neutralizing activity of LSC-Exo against SARS-CoV-2 pseudovirus, ELISA-based blocking assay was used to confirm the specific binding of RBD with rhACE2 was inhibited by LSC-Exo in a dose-dependent manner, unlike the ACE2-deficient HEK-Exo (FIG. 28A), indicating that LSC-Exo have a stronger binding ability to RBD. To analyze the efficacy of LSC-Exo on viral attachment and infection, a SARS-CoV-2 pseudovirus-based assay was implemented assessing the protective activity of LSC-Exo to A549 cells expressing ACE2 receptor (FIG. 28B). In a dose-dependent manner, LSC-Exo efficiently intercepted the entry of SARS-CoV-2 pseudovirus with an GFP reporter into ACE2-expressing A549 cells (FIG. 28C). In contrast, an equal amount of HEK-Exo had negligible inhibition effects, whereas the positive control, rhACE2, efficiently blocked the infection of SARS-CoV-2 pseudovirus in A549 cells. Flow cytometry (FIG. 28D) further validated that LSC-Exo and rhACE2 efficiently neutralized SARS-CoV-2 pseudoviruses and prevented them entry into host cells, while HEK-Exo failed to inhibit this entry.

Having demonstrated that LSC-Exo were able to neutralize SARS-CoV-2 at the cellular level, additional experiments were conducted to evaluate their neutralization ability in vivo. On the basis of the results of LSC-Exo's biodistribution in vivo, the mice were nebulized with LSC-Exo 2 hours before SARS-CoV-2 pseudovirus challenge. Ex-vivo fluorescence imaging showed that substantial pseudovirus signals were detected in the mice treated with HEK-Exo (FIG. 28E). Conversely, dim pseudovirus signals were observed in the mice treated with LSC-Exo, indicative of successful inhibition of virus entry. Intriguingly, rhACE2 failed to block SARS-CoV-2 pseudovirus entry into mice (FIG. 28F), which might be attributed to the rapid clearance of free rhACE2 in vivo. Whole lung imaging further confirmed that fewer SARS-CoV-2 pseudoviruses were distributed in both the trachea/bronchioles and parenchyma in the mice with LSC-Exo treatment, rather than HEK-Exo or rhACE2 treatment (FIG. 28G) Collectively, those compound datasets suggested that LSC-Exo are capable of neutralizing the SARS-CoV-2 pseudovirus and preventing their infection to the host cells.

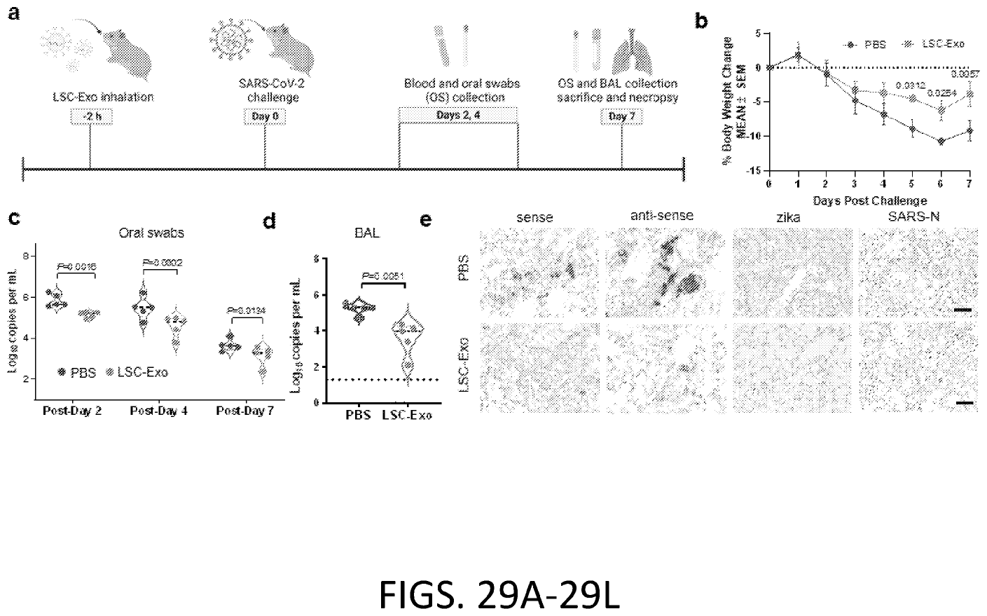

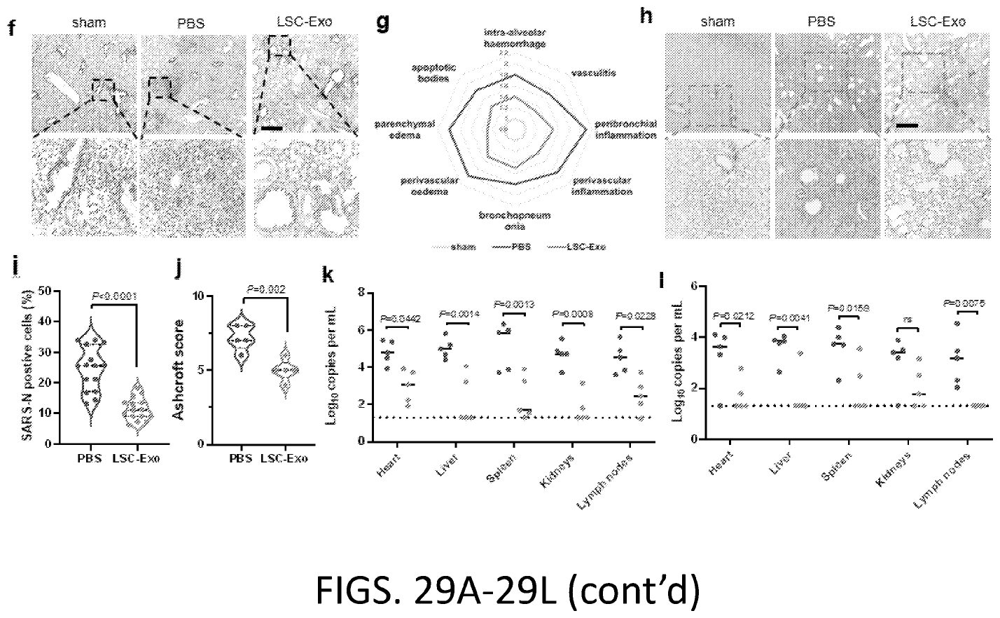

LSC-Exo protect Syrian hamsters from SARS-CoV-2 infection. The Syrian golden hamsters could capture the diverse pathologies of SARS-CoV-2 infection, who were thus employed to evaluate the prophylactic capacity of LSC-Exo against SARS-CoV-2 infection. Inhalation of LSC-Exo at 2 hours before challenging with live SARS-CoV-2 significantly prevented SARS-CoV-2-induced weight loss as compared to PBS treatment (FIGS. 29A-29B). Moreover, this protection was associated with decreased viral load in both oral swabs (OS) and bronchoalveolar lavage (BAL) of hamsters (FIGS. 29C-29D). In situ RNA hybridization analysis further revealed that LSC-Exo prophylaxis resulted in less viral RNA presented in the lung tissues of hamsters compared with PBS treatment (FIG. 29E). Immunohistochemistry (IHC) staining for nucleocapsid (N) protein of the SARS-CoV-2 (SARS-N) indicated that viral protein in the lung tissues was reduced by LSC-Exo treatment relative to PBS control (FIGS. 29E and 29I). Examination of lung tissues from infected hamsters with PBS treatment revealed swollen alveolar lining cells, remarkable inflammatory infiltrates filled with large numbers of neutrophils, macrophages, and lymphocytes in the alveolar walls and air spaces (FIGS. 29F and 29G). Conversely, LSC-Exo treatment greatly reduced the severity and incidence of alveolar infiltration and interstitial pneumonia in hamsters. Masson's trichrome staining and Ashcroft score exhibited that LSC-Exo significantly dampened lung fibrosis with the preservation of alveolar epithelial structures as compared to PBS treatment (FIGS. 29H and 29J). Finally, it was observed that both viral genomic RNA levels (FIG. 29K) and subgenomic RNA (sgRNA) levels (FIG. 29L) in the heart, liver, spleen, kidneys, and lymph nodes tissues were greatly decreased in hamsters that received LSC-Exo treatment, not PBS control, indicating that LSC-Exo could protect the distant tissues of hamsters against SARS-CoV-2 infection, including but not limited to protecting the lung.

Taken together, these data identified the expression of ACE2 receptor on healthy buman lung spheroid cells (LSC) and LSC-derived exosomes (LSC-Exo) and compared them with ACE2-deficient HEK cells and HEK-Exo. Results demonstrated that LSC-Exo carried much more hACE2 receptors than HEK-Exo, as verified by immunoblot analysis and flow cytometry measurements. In vitro analysis showed that LSC-Exo were able to prevent the entry of SARS-CoV-2 pseudovirus into the ACE2 receptor-expressing A549 cells, similar to free rhACE2, whereas HEK-Exo showed little neutralization capacity against SARS-CoV-2 pseudovirus. Significantly, it was found that LSC-Exo exhibited a higher inhibitory activity against SARS-CoV-2 pseudovirus than rhACE2 in vivo, which could be attributed to the rapid degradation and clearance of free rhACE2 in physiological environment. Furthermore, LSC-Exo were demonstrated to be more effective in evading mucoadhesion and directly delivering to the respiratory system over the Lipo counterpart by nebulization, suggesting that LSC-Exo have enhanced cellular targeting within the lung due to exosome phenotypes that are native to the lung microenvironment.

As described herein, experiments were conducted to evaluate the prophylactic efficacy of LSC-Exo in SARS-CoV-2-infected Syrian hamsters, which can recapitulate serious COVID-19 diseases. Results demonstrated that inhalation of LSC-Exo significantly interrupted the interaction of S protein with entry receptor ACE2 and efficiently protected the hamsters against SARS-CoV-2 infection. In contrast to PBS group, LSC-Exo significantly decreased the viral replication as demonstrated by reduced viral load in major organ tissues of hamsters (heart, liver, spleen, lung, kidneys, and lymph nodes). Lung examinations revealed that hamsters which were inhaled LSC-Exo did not exhibit fulminant pulmonary disease as observed in hamsters treated with PBS. RNA-Seq analysis provided direct evidence that LSC-Exo not only were able to efficiently reduce immune activation, maintain intracellular ROS homeostasis, and dampen inflammatory cytokine storm, but also alleviated pulmonary dysfunction of the hamsters by activating the antioxidant defense systems. Despite the emergence of SARS-CoV-2 VOC intensively decreased the effectiveness of current vaccines and neutralizing antibodies, these data demonstrated that LSC-Exo retain potent neutralization activity for all variant pseudoviruses examined, efficiently intercepting the VOC pseudoviruses entry into the lung of mice.

What is claimed is:

1. A composition comprising a plurality of engineered extracellular vesicles (EVs), wherein the plurality of EVs comprise:
(i) at least one membrane-associated protein on the surface of the plurality of EVs; and/or
(ii) at least one therapeutic agent loaded into the plurality of EVs.

2. The composition of claim 1, wherein the plurality of EVs are derived from a cell.

3. The composition of claim 1 or claim 2, wherein the plurality of EVs are derived from a cell selected from the group consisting of: HeLa cells, HEK293 cells, HEK293 derived cells, Vero cells, CHO cells, CHO-K1 cells, CHO-derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, and RK cells.

4. The composition of claim 1 or claim 2, wherein the plurality of EVs are derived from a lung spheroid cell (LSC).

5. The composition of any of claims 1 to 4, wherein the plurality of EVs comprise liposomes.

6. The composition of any of claims 1 to 4, wherein the plurality of EVs comprise exosomes.

7. The composition of any of claims 1 to 6, wherein the plurality of EVs are from about 30 nm to about 1000 nm in diameter.

8. The composition of any of claims 1 to 7, wherein the plurality of EVs comprise an average size from about 100 nm to about 200 nm in diameter.

9. The composition of any of claims 1 to 8, wherein the at least one membrane-associated protein on the surface of the plurality of EVs comprises a viral-specific protein, or a derivative or fragment thereof.

10. The composition of claim 9, wherein the viral-specific protein comprises an antigenic epitope or derivative or fragment thereof capable of stimulating an immune response in a subject

11. The composition of claim 9, wherein the viral-specific protein comprises a coronavirus Spike protein (S protein), or a derivative or fragment thereof.

12. The composition of claim 9, wherein the viral-specific protein comprises a receptor binding domain (RBD) of a coronavirus Spike protein (S protein), or a derivative or fragment thereof, capable of binding Angiotensin-converting enzyme 2 (ACE2).

13. The composition of any of claims 1 to 12, wherein the at least one therapeutic agent loaded into the plurality of EVs comprises mRNA encoding an antigenic epitope or derivative or fragment thereof capable of stimulating an immune response in a subject.

14. The composition of claim 13, wherein the mRNA encodes an antigenic epitope or a derivative or fragment thereof capable of stimulating an immune response in a subject from a coronavirus Spike protein (S protein), or a derivative or fragment thereof.

15. The composition of claim 13, wherein the mRNA encodes an antigenic epitope or a derivative or fragment thereof capable of stimulating an immune response in a subject from a receptor binding domain (RBD) of a coronavirus Spike protein (S protein), or a derivative or fragment thereof, capable of binding Angiotensin-converting enzyme 2 (ACE2).

16. The composition of any of claims 1 to 8, wherein the at least one therapeutic agent loaded into the plurality of EVs comprises at least one mRNA encoding a tumor-associated antigen (TAA).

17. The composition of claim 16, wherein the TAA is selected from the group consisting of: MAGE-C1, MAGE-C2, MAGE-C3, MAGE-A3, NY-SEO-1, survivin, 5 T4, MUC1, PSA, PSCA, PSMA, STEAP1, PAP, tyrosinase, GP100, and CT7.

18. The composition of any of claims 1 to 8, wherein the at least one therapeutic agent loaded into the plurality of EVs comprises at least one mRNA encoding an immunostimulant.

19. The composition of claim 18, wherein the immunostimulant is selected from the group consisting of: IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, INF-gamma, GM CSF, G-CSF, M-CSF, LT-βor TNF-α, OX40L, CD40L, and CD7.

20. The composition of claim 18, wherein the immunostimulant is IL-12.

21. The composition of any of claims 1 to 8, wherein the at least one therapeutic agent loaded into the plurality of EVs comprises at least one small molecule.

22. The composition of claim 21, wherein the at least one small molecule is an anti-cancer drug.

23. The composition of claim 22, wherein the anti-cancer drug is selected from the group consisting of: a kinase inhibitor, an ALK inhibitor, a c-Met inhibitor, an EGFR inhibitor, an FLT3 inhibitor, a VEGFR/FGFR/PDGFR inhibitor, a TRK inhibitor, Bcr-Abll inhibitor, a BTK inhibitor, a JAK inhibitor, a BRAF/MEK/ERK inhibitor, a CDK inhibitor, a PI3K/AKT/mTOR inhibitor, an EZH2 inhibitor, an HDAC inhibitor, an IDH1/2 inhibitor, and a BCL-2 inhibitor.

24. The composition of claim 22, wherein the anti-cancer drug is a VEGFR/FGFR/PDGFR inhibitor selected from the group consisting of: nintedanib, sorafenib, sunitinib, lenvatinib, pazopanib, axitinib, cabozantinib, tivozanib, apatinib, anlotinib, fruquintinib, erdafitinib, pemigatinib, avapritinib, imatinib, regorafenib, ripretinib, cediranib, dovitinib, motesanib, crenolanib, lucitanib, vactosertib, vandetanib, selpercatinib, pralsetinib, sulfatinib, and brivanib.

25. The composition of any of claims 1 to 24, wherein loading of the at least one therapeutic agent comprises encapsulating the at least one therapeutic agent in the EV membrane and/or encapsulating the at least one therapeutic agent within the lumen of the EV.

26. The composition of any of claims 1 to 25, wherein the composition further comprises at least one pharmaceutically-acceptable excipient or carrier.

27. A method of preventing and/or treating a viral infection comprising administering any of the compositions of any of claims 1 to 15 to a subject.

28. The method of claim 27, wherein the virus is a coronavirus.

29. The method of claim 28, wherein the coronavirus is selected from the group consisting of 229E, NL63, OC43, HKU1, MERS-CoV, SARS-CoV, and SARS-CoV-2, or any variants thereof.

30. A method of treating cancer comprising administering any of the compositions of any of claims 1 to 8 and claims 16 to 25 to a subject.

31. The method of any of claims 27 to 30, wherein the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration.

32. The method of any of claims 27 to 30, wherein the composition is administered via nebulization to lung tissue.
